(19) United States
(12) Patent Application Publication
(10) Pub. No.: US 2025/0161309 A1
(43) Pub. Date: May 22, 2025

(54) TOPICAL COMPOSITIONS AND USES THEROF

(71) Applicant: TOPADUR PHARMA AG, Schlieren (CH)

(72) Inventors: Reto NAEF, Rheinfelden (CH); Paola ATZEI, Schlieren (CH); Rita FRANCISCO, Zürich (CH)

(21) Appl. No.: 18/841,618

(22) PCT Filed: Mar. 1, 2023
(86) PCT No.: PCT/EP2023/055085

§ 371 Date: Aug. 26, 2024

(30) Foreign Application Priority Data
Mar. 2, 2022 (EP) 22159773.5

(51) Int. Cl. A61K 31/519; A61K 9/00; A61K 47/10; A61P 17/14

(52) U.S. Cl. CPC A61K 31/519; A61K 9/0014; A61K 47/10; A61P 17/14

(57) ABSTRACT

The present invention relates to a topical composition comprising a compound of formula (I) or a pharmaceutically acceptable salt thereof wherein R₁ is C₁-C₃alkyl, preferably methyl or ethyl, further preferably ethyl; R₂ is H, CHO or CH═N—OH, preferably H, CHO or (E)-CH═N—OH, further preferably H; R₃ is C₁-C₄alkyl, preferably ethyl or propyl, further preferably n-propyl; R₄ is C₁-C₆alkyl, preferably ethyl or propyl, further preferably n-propyl; R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said R₁₅ comprises at least one ONO₂ moiety; wherein said topical composition comprises said compound of formula (I), preferably said Compound 1, from about 0.0001% (w/v) to about 0.05% (w/v); as well as to the uses of said topical compositions for the treatment of diseases or disorders mediated by PDE5 activity and/or NO related endothelial dysfunction, preferably of a disease or disorder of the skin mediated by PDE5 activity and/or NO related endothelial dysfunction, in a subject, preferably in a human, and particularly, for topically treating a skin disease, preferably skin aging or steroid-induced skin atrophy, erectile dysfunction, preventing or treating hair loss, or promoting hair growth.

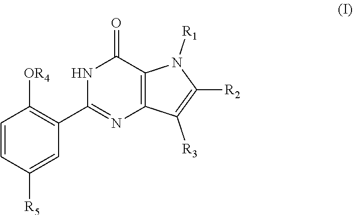

### TOPICAL COMPOSITIONS AND USES THEREOF

The present invention relates to topical compositions comprising compounds of formula I or a pharmaceutically acceptable salt thereof. The invention also relates to uses of said topical compositions for the treatment of diseases or disorders alleviated by inhibition of PDE5 and/or NO related endothelial dysfunction in a subject, preferably in a human, and particularly, for topically treating a skin disease, preferably skin aging or steroid-induced skin atrophy, erectile dysfunction, preventing or treating hair loss, or promoting hair growth.

### RELATED ART

Phosphodiesterases (PDEs) are enzymes that catalyzes the hydrolysis and thus the degradation of cyclic adenosine monophosphate (cAMP) and cyclic guanosine monophosphate (cGMP) and thereby regulates intracellular levels of second messengers. Inhibition of PDEs leads to increasing intracellular concentrations of endogenous cAMP/cGMP. Therefore, inhibition of PDE can mediate a variety of physiological mechanisms at different cell and organ levels. Phosphodiesterase type 5 (PDE5) hydrolyses cyclic guanylate monophosphate (cGMP) specifically to 5′ GMP. The selective inhibition of PDE5 has been validated as a relevant approach and strategies directed to promote inhibition of PDE5 activity have been applied as therapeutic tools, in particular, in neuronal, vascular and cardiovascular diseases and disorders. Moreover, the introduction of PDE5 inhibitors has revolutionized the treatment of male erectile dysfunction (Andersson K E, British Journal of Pharmacology (2018) 175:2554-2565; Das A et al. Pharmacol Ther. (2015) 147:12-21; Dobhal T et al. Critical Review in Pharmaceutical Sciences (2012) 1(3):13-27). Most prominent examples of PDE5 inhibitors are Sildenafil, Tadalafil and Vardenafil, which have been described among others, for example, in WO 99/24433, WO 01/60825, EP 995′751 and WO 2011/075655. Recently a novel class of PDE 5 inhibitors with dual-pharmacology releasing NO in addition to its PDE 5 inhibition in a more than additive fashion has been described (WO/2018/215433).

Hair loss is a symptom of abnormally falling out of a lot of hair. Although there are many cases of hair falling out, abnormal hair loss of beard, eyebrows, public hair, armpit hair and other areas is also referred to as hair loss. Hair is produced in hair follicles, which are located in the dermis layer above the subcutaneous fat below the epidermal layer. Hair follicles are located on all parts of the body except the lips, palms and soles of the feet, and new hair is made from the hair matrix at the bottom of the hair follicles. The living cells in the hair matrix proliferate and push upward, and when these cells dry quickly and die, they are compressed into a dense and hard mass to form a hair shaft. The hair shaft, which is made of dead protein, is covered with a delicate layer (cuticula pili) composed of plate-like scales.

The hair growth cycle consists of three phases: the anagen phase, where hair grows most actively, followed by the catagen phase, where hair degeneration begins, and the telogen phase, where hair growth is stopped. At the end of the telogen phase, the hair falls out and a new cycle begins again as new hair grows from the hair follicles. The anagen phase of eyebrows and eyelashes is about 1 to 6 months, and the anagen phase of hair is about 2 to 6 years, and in general, about 50 to 100 hairs fall out at the end of the telogen phase each day.

The most common type of male hair loss is male pattern baldness or alopecia, which develops gradually over several years and begins most prominently on the crown of the head and progresses to the frontal region. In the case of female hair loss, it occurs in a more dispersed form as the thickness of the hair becomes thinner and often occurs after menopause.

Many studies are being conducted to alleviate or treat alopecia, and in particular, research has been carried out in the cosmetic or pharmaceutical industry for a long time to develop new substances for stimulating hair growth or alleviating hair loss, and accordingly, drug therapy using female hormones, blood flow promoters and the like and hair transplantation have been developed. Currently, the most commonly used drugs for the treatment of hair loss are 2,4-diamino-6-piperidinopyrimidine-3-oxide (also known as “Minoxidil” formulation, refer to U.S. Pat. Nos. 4,193,619 and 4,596,812), which has been approved by the FDA, and finasteride, which is a specific inhibitor of type II 5α-reductase.

Minoxidil formulation is a drug that induces hair growth by increasing blood flow through the vasodilating effect and supplying nutrients to the hair root, and in particular, it is known to be effective for alleviating hair loss symptoms in the forehead area, and pharmaceutical products using the same as an active ingredient are marketed under the trade name Rogaine (trade name of Pharmacia & Upjohn Company). Rogaine is known to reduce hair loss by up to 10% and promote hair growth in men suffering from male pattern baldness. Rogaine has to be used regularly for a long period of time and it does not obviously exert a good effect on hair loss in areas other than the forehead area.

Pharmaceutical products using finasteride as an active ingredient are marketed under the trade name Propecia (trade name of Propecia, Merck & Co., Inc.), and it is known as a pill for oral administration to inhibit hair loss by inhibiting the function of type II 5α-reductase and preventing the conversion of testosterone to dihydrotestosterone (DHT). Propecia requires continuous and regular administration, and for some patients, it has side effects such as decreased libido, erectile dysfunction and the like. Propecia can be used only for adult men.

Thus, there is still a medical need for treatment of a disease or disorder mediated by PDE5 activity and/or NO related endothelial dysfunction, in particular for preventing or treating hair loss and alopecia as well as for promoting hair growth.

### SUMMARY OF THE INVENTION

The inventors have surprisingly found that topical treatment of the compounds and compositions of the invention demonstrated excellent therapeutic effect not only for male pattern baldness but also for female pattern baldness and improve hair growth in in vivo experiments and exhibit a remarkably excellent effect even in hair loss induced by chemotherapy used for cancer treatment and the like. The excellent therapeutic effects have been shown, in particular, as compared to Minoxidil which is one of the most currently used drugs and available in the market. Moreover, it was shown that hair loss could be prevented when the compounds and compositions of the invention were administered before actual hair loss occurred. Notably, the excellent therapeutic effects obtained by the compounds and compositions of the invention required remarkably low amounts and concentrations of the inventive compounds and compositions, respectively.

Thus, in a first aspect, the present invention provides a topical composition comprising a compound of formula I, preferably an effective amount of a compound of formula I, or a pharmaceutically acceptable salt thereof

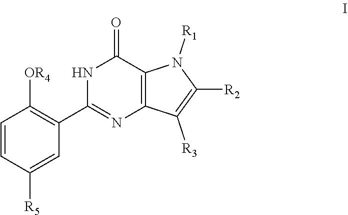

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein preferably said topical composition comprises said compound of formula I, preferably said Compound 1, from about 0.0001% (w/v) to about 0.05% (w/v). Thus, preferably, said compound of formula I, preferably said Compound 1, is present in said topical composition from about 0.0001% (w/v) to about 0.05% (w/v).

In another aspect, the present invention provides a topical composition comprising a compound of formula I, preferably an effective amount of a compound of formula I, or a pharmaceutically acceptable salt thereof

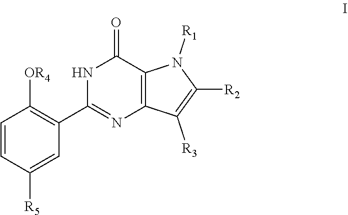

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein said topical composition comprises said compound of formula I, preferably said Compound 1, from about 0.0001% (w/v) to about 0.05% (w/v). Thus, said compound of formula I, preferably said Compound 1, is present in said topical composition from about 0.0001% (w/v) to about 0.05% (w/v).

In another aspect, the present invention provides a topical composition comprising
(a) a compound of formula I, preferably an effective amount of a compound of formula I, or a pharmaceutically acceptable salt thereof

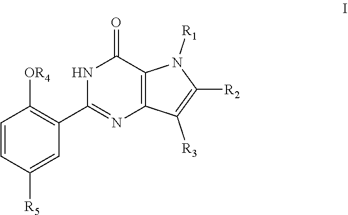

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein said topical composition comprises said compound of formula I, preferably said Compound 1, from about 0.0001% (w/v) to about 0.05% (w/v). Thus, said compound of formula I, preferably said Compound 1, is present in said topical composition from about 0.0001% (w/v) to about 0.05% (w/v).
(b) a solvent; and
(c) optionally one or more other pharmaceutically acceptable excipients.

The topical composition of the present invention can be used for the topical treatment of diseases or disorders of the skin being mediated by or resulting from PDE5 activity and/or NO related endothelial dysfunction. Moreover, the topical composition of the present invention can be used for the topical treatment of diseases or disorders being mediated by or resulting from PDE5 activity and/or NO related endothelial dysfunction. Furthermore, the topical composition of the present invention can be used for the topical treatment of diseases or disorders of the skin being mediated by or resulting from PDE5 activity and/or NO related endothelial dysfunction, In particular, the topical composition of the present invention can be used for the topical treatment of diseases or disorders in a subject, preferably in a human, wherein said topically treating of said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

Thus, in a further aspect, the present invention provides the topical composition of the present invention for use in a method of topically treating a disease or disorder mediated by PDE5 activity and/or NO related endothelial dysfunction, preferably of a disease or disorder of the skin mediated by PDE5 activity and/or NO related endothelial dysfunction, in a subject, preferably in a human. Preferably, said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides the topical composition of the present invention for use in a method of topically treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides a method for treating a disease or disorder mediated by PDE5 activity and/or NO related endothelial dysfunction, preferably of a disease or disorder of the skin mediated by PDE5 activity and/or NO related endothelial dysfunction, in a subject, preferably in a human, wherein said method comprises topically administering the topical composition of the present invention to said subject, preferably to said human.

In another aspect, the present invention provides a method for treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle, wherein said method comprises topically administering the topical composition of the present invention to said subject, preferably to said human. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides for the use of the topical composition of the present invention for the preparation of a medicament for topically treating a disease or disorder mediated by PDE5 activity and/or NO related endothelial dysfunction, preferably of a disease or disorder of the skin mediated by PDE5 activity and/or NO related endothelial dysfunction, in a subject, preferably in a human, wherein said method comprises topically administering the topical composition of the present invention to said subject, preferably to said human.

In another aspect, the present invention provides for the use of the topical composition of the present invention for the preparation of a medicament for topically treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle, wherein said method comprises topically administering the topical composition of the present invention to said subject, preferably to said human. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle Further aspects and embodiments of the present invention will be become apparent as this description continues.

### DESCRIPTION OF FIGURES

FIG. 1: A mimetic diagram of an in vivo mouse model experiment for confirming the hair loss treatment effect of Cpd 1 and Cpd 2 dissolved in PEG-400:EtOH/7:3

FIG. 2A and FIG. 2B: Result of comparing the weight change (FIG. 2A) and hair growth effect (FIG. 2B) according to the dose-specific application of Cpd 1 dissolved in PEG-400:EtOH/7:3 with a Minoxidil application in male mice. In FIG. 2B a, b indicate p<0.05 and p<0.01 respectively when Cpd 1 and Minoxidil were compared to vehicle group.

FIG. 3A and FIG. 3A: A set of photographs comparing the hair anagen phase inducing effect (FIG. 3A) and the melanin production effect (FIG. 3B—visual melanogenesis) according to the dose-specific application of Cpd 1 dissolved in PEG-400:EtOH/7:3 to a Minoxidil application in male C57BL/6 mice.

FIG. 4A, FIG. 4B and FIG. 4C: Result of comparing the transverse skin sections (FIG. 4A), the number of hair follicles (FIG. 4B) and the skin thickness (FIG. 4C) according to the dose-specific application of Cpd 1 dissolved in PEG-400:EtOH/7:3 to a Minoxidil application by H&E staining in male mice.

FIG. 5A and FIG. 5B: Result of comparing the weight change (FIG. 5A) and hair growth effect (FIG. 5B) according to the dose-specific application of Cpd 1 dissolved in PEG-400:EtOH/7:3 compared to a Minoxidil application in in female mice. In FIG. 5B a, b, c indicate p<0.05 p<0.01 and p<0.001 respectively when Cpd 1 groups were compared to vehicle group.

FIG. 6A and FIG. 6B: A set of photographs comparing the hair anagen phase inducing effect (FIG. 6A) and the melanin production effect (FIG. 6B—visual melanogenesis) according to the dose-specific application of Cpd 1 dissolved in PEG-400:EtOH/7:3 to a Minoxidil application in female C57BL/6 mice.

FIG. 7A, FIG. 7B and FIG. 7C: Result of comparing the transverse skin sections (FIG. 7A), the number of hair follicles (FIG. 7B) and the skin thickness (FIG. 7C) according to the dose-specific application of Cpd 1 dissolved in PEG-400:EtOH/7:3 to a Minoxidil application by H&E staining in female mice.

FIG. 8A and FIG. 8B: Result of comparing the weight change (FIG. 8A) and hair growth effect (FIG. 8B) according to the dose-specific application of Cpd 2 dissolved in PEG-400:EtOH/7:3 with a Minoxidil application in female mice. In FIG. 8B a, b, c, d indicate p<0.05, p<0.01, p<0.001 and p<0.0001 respectively when Cpd 2 groups were compared to vehicle; e indicates p<0.05, when 0.02% Cpd 2 groups was compared to 2% Minoxidil.

FIG. 9A and FIG. 9A: A set of photographs comparing the hair anagen phase inducing effect (FIG. 9A) and the melanin production effect (FIG. 9B—visual melanogenesis) according to the dose-specific application of Cpd 2 dissolved in PEG-400:EtOH/7:3 to a Minoxidil application in female C57BL/6 mice.

FIG. 10A, FIG. 10B and FIG. 10C: Result of comparing the transverse skin sections (FIG. 10A), the number of hair follicles (FIG. 10B) and the skin thickness (FIG. 10C) according to the dose-specific application of Cpd 2 dissolved in PEG-400:EtOH/7:3 to a Minoxidil application by H&E staining in female mice.

FIG. 11: A mimetic diagram of an in vivo mouse model experiment for confirming the effect of treating hair loss by Cpd 1 dissolved in PEG-400:EtOH/7:3 caused by chemotherapy.

FIG. 12A, FIG. 12B and FIG. 12C: Result of comparing the body weight (FIG. 12A), hair growth effect (FIG. 12B) and hair weight (FIG. 12C) according to the dose-specific application RECTIFIED SHEET (RULE 91) ISA/EP of Cpd 1 dissolved in PEG-400:EtOH/7:3 with a Minoxidil application. In FIG. 12B a, b, c, d indicate p<0.05, p<0.01, p<0.001 and p<0.0001 respectively when Cpd 1 groups were compared to vehicle; e, f g, h indicate p<0.05, p<0.01, p<0.001 and p<0.0001 respectively, when Cpd 1 groups were compared to 2% Minoxidil group. In FIG. 12C ***, **** and ns indicate p<0.001, p<0.0001 and non-significant respectively when comparing pre- and post-treatment animals from the same group.

FIG. 13A and FIG. 13B: A set of photographs comparing the hair anagen phase inducing effect (FIG. 13A) and the melanin production effect (FIG. 13B—visual melanogenesis) according to the dose-specific application of Cpd 1 dissolved in PEG-400:EtOH/7:3 to a Minoxidil application.

FIG. 14A, FIG. 14B and FIG. 14C: Result of comparing the transverse skin and longitudinal skin sections (FIG. 14A), the number of hair follicles (FIG. 14B) and the skin thickness (FIG. 14C) according to the dose-specific application of Cpd 1 dissolved in PEG-400:EtOH/7:3 to a Minoxidil application by H&E staining in female mice.

FIG. 15A, FIG. 15B, FIG. 15C and FIG. 15D: Result of comparing the expressions of the CD-31 angiogenesis marker (FIG. 15A, FIG. 15B) and Ki-67 cell proliferation marker (FIG. 15C, FIG. 15D) according to the dose-specific application of Cpd 1 dissolved in PEG-400:EtOH/7:3 with Minoxidil

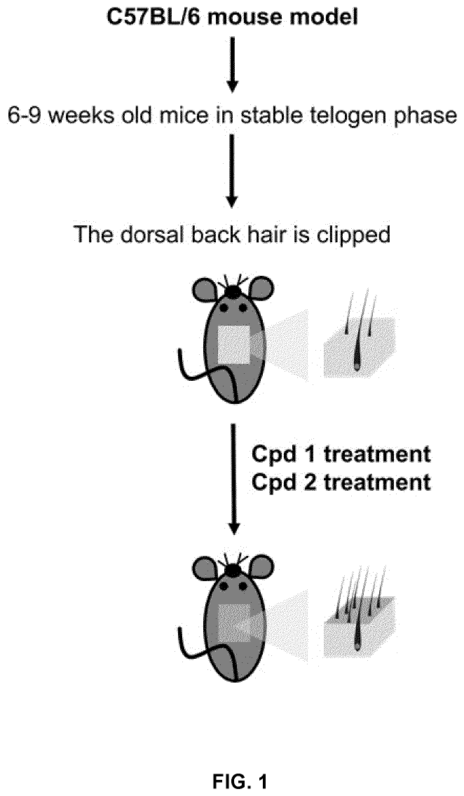

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to.

The term “about”, as used herein shall have the meaning of +/−10%. For example about 50% shall mean 45% to 55%. Preferably, the term “about”, as used herein shall have the meaning of +/−5%. For example about 50% shall mean 47.5% to 52.5%.

The phrase “between number X and number Y”, as used herein, shall refer to include the number X and the number Y. For example, the phrase “between 0.01 μmol and 50 μmol” refers to 0.01 μmol and 50 μmol and the values in between. The same applies to the phrase “between about number X and about number Y”.

The term “% (v/v)” as used herein refers to (volume of the component/total volume of the composition)×100. By way of example, 70% (v/v) PEG400 refers to 70 ml PEG400 per 100 ml of the total composition.

When the terms “a,” or “an” are used herein, they mean “at least one” unless indicated otherwise.

As used herein, the term “topically” and “topical” refers to application of the composition of the present invention to the surface of the skin of a subject, preferably of a human, and thus, the term “topical administration”, as used herein, refers to administration to the skin of a subject, preferably of a human, preferably to prevent or treat hair loss, wherein said hair loss is alopecia, and wherein preferably said alopecia is androgenetic alopecia (AGA) or chemotherapy-induced alopecia (CIA) or alopecia areata; or to promote hair growth.

As used herein the term “topical composition” refers to a composition that is suitable for topical administration and may be applied to skin of a subject, preferably of a human.

As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment. Preferably, beneficial or desired clinical results of said treatment include, but are not limited to, alleviation of symptoms, diminishment of extent of disease or disorder, stabilized (i.e., not worsening) state of disease or disorder, delay or slowing of disease or disorder progression, amelioration or palliation of the disease or disorder state.

As used herein, the term “effective amount” refers to an amount necessary or sufficient to realize a desired biologic effect. Preferably, the term “effective amount” refers to an amount of a compound of formula I of the present invention that (i) treats or prevents the particular disease or disorder, (ii) attenuates, ameliorates, or eliminates one or more symptoms of the particular disease or disorder, or (iii) prevents or delays the onset of one or more symptoms of the particular disease or disorder, described herein. An effective amount of the inventive compound of formula I, or said topical composition or said pharmaceutical composition, would be the amount that achieves this selected result, and such an amount could be determined as a matter of routine by a person skilled in the art. Further preferably, the term “effective amount”, as used herein, refers to an amount necessary or sufficient to be effective to activation of soluble guanylyl cyclase (sGC) and/or increase the inhibition of PDE5. The effective amount can vary depending on the particular composition being applied and the size of the subject. One of ordinary skill in the art can empirically determine the effective amount of a particular the inventive compound of formula I, or said topical composition or said pharmaceutical composition of the present invention without necessitating undue experimentation.

The term “mammal”, as used herein, includes, but is not limited to, humans, mice, rats, guinea pigs, monkeys, dogs, cats, horses, cows, pigs, and sheep. The term “mammal”, as used herein, preferably refers to humans. The term “subject”, as used herein, includes, but is not limited to, humans and mammals. The term “subject”, as used herein, preferably refers to humans.

Thus, in a first aspect, the present invention provides a topical composition comprising a compound of formula I, preferably an effective amount of a compound of formula I, or a pharmaceutically acceptable salt thereof

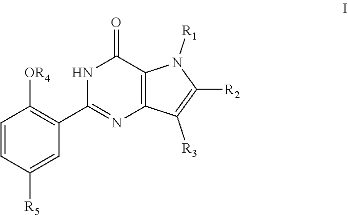

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein preferably said topical composition comprises said compound of formula I, preferably said Compound 1, from about 0.0001% (w/v) to about 0.05% (w/v). Thus, preferably, said compound of formula I, preferably said Compound 1, is present in said topical composition from about 0.0001% (w/v) to about 0.05% (w/v).

In another aspect, the present invention provides a topical composition comprising a compound of formula I, preferably an effective amount of a compound of formula I, or a pharmaceutically acceptable salt thereof

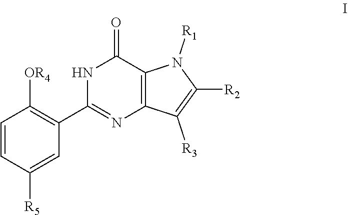

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein said topical composition comprises said compound of formula I, preferably said Compound 1, from about 0.0001% (w/v) to about 0.05% (w/v). Thus, said compound of formula I, preferably said Compound 1, is present in said topical composition from about 0.0001% (w/v) to about 0.05% (w/v).

In another aspect, the present invention provides a topical composition comprising (a) a compound of formula I, preferably an effective amount of a compound of formula I, or a pharmaceutically acceptable salt thereof

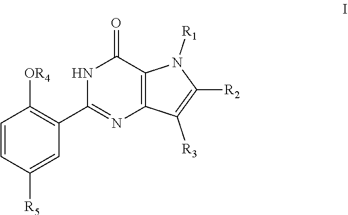

wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety;
wherein said topical composition comprises said compound of formula I, preferably said Compound 1, from about 0.0001% (w/v) to about 0.05% (w/v). Thus, said compound of formula I, preferably said Compound 1, is present in said topical composition from about 0.0001% (w/v) to about 0.05% (w/v).
(b) a solvent; and
(c) optionally one or more other pharmaceutically acceptable excipients.

Compounds of formula I comprised in the topical composition of the present invention include pharmaceutically acceptable salts of said compounds. In particular, the term “pharmaceutically acceptable salt” as used herein, refers to pharmaceutically acceptable organic or inorganic salts of a compound of the present invention, in particular acid addition salts. Exemplary salts include, but are not limited to, salts of physiologically acceptable mineral acids, such as hydrochloric acid, sulfuric acid, nitric acid and phosphoric acid, or salts of organic acids, such as methane-sulfonic acid, p-toluenesulfonic acid, lactic acid, malic acid, tartaric acid, acetic acid, trifluoroacetic acid, citric acid, succinic acid, fumaric acid, maleic acid and salicylic acid. Further examples of pharmacologically acceptable salts of the compounds of formula I are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts. Further examples of pharmaceutically acceptable salts of the compounds of formula I include the hydrochloride, hydrobromide, sulfate, bisulfate, phosphate, hydrogen phosphate, nitrate, acetate, benzoate, succinate, fumarate, maleate, lactate, citrate, benzenesulphonate, p-toluenesulphonate or the like.

In a preferred embodiment, said R₁ is methyl or ethyl. In a preferred embodiment, said R₁ is methyl. In a preferred embodiment, said R₁ is ethyl. In another preferred embodiment, said R₂ is H. In a further preferred embodiment, said R₃ is ethyl or n-propyl. In a further preferred embodiment, said R₃ is ethyl. In a further preferred embodiment, said R₃ is n-propyl. In another preferred embodiment, said R₄ is ethyl or n-propyl. In another preferred embodiment, said R₄ is ethyl. In another preferred embodiment, said R₄ is n-propyl. In an again further preferred embodiment, said R₁ is methyl or ethyl; said R₂ is H; said R₃ is ethyl or n-propyl; and said R₄ is ethyl or n-propyl. In an again further preferred embodiment, said R₁ is ethyl; said R₂ is H; said R₃ is n-propyl; and said R₄ is n-propyl.

In a preferred embodiment, said R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said at least one R₁₅ comprises at least one ONO₂ moiety. In a preferred embodiment, said heterocyclic ring is piperidine. In another preferred embodiment, said heterocyclic ring is piperazine. In a preferred embodiment, said R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with one, two, three, four, five of six R₁₅, and wherein said R₁₅ is independently selected from C₁-C₆alkyl optionally substituted with OH, ONO₂, wherein at least one, preferably one, two, three or four of said at least one R₁₅ comprises at least one ONO₂ moiety. In a preferred embodiment, said R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with one, two or three R₁₅, and wherein said R₁₅ is independently selected from C₁-C₆alkyl optionally substituted with OH, ONO₂, wherein at least one, preferably one, two or three of said at least one R₁₅ comprises at least one ONO₂ moiety.

In a preferred embodiment, said compound of formula I is a compound of formula I* or a compound of formula I**, or pharmaceutically acceptable salt, solvate or hydrate thereof,

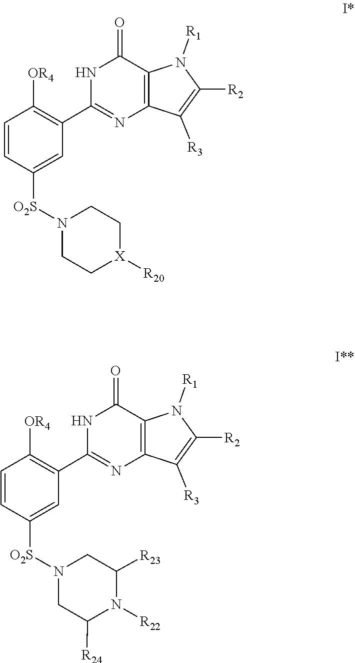

wherein R₁, R₂, R₃, and R₄, are as defined herein including all preferred and very preferred embodiments of R₁, R₂, R₃, and R₄ alone or in combination, and wherein
X is CR₂₁ or N;
R₂₀ is C₁-C₆alkyl optionally substituted with OH and/or ONO₂;
R₂₁ is H or C₁-C₆alkyl optionally substituted with OH and/or ONO₂;
   wherein at least one of said R₂₀ and R₂₁ comprises at least one ONO₂ moiety;
R₂₂ is H or C₁-C₆alkyl optionally substituted with OH and/or ONO₂;
R₂₃ and R₂₄ are each independently C₁-C₆alkyl optionally substituted with OH and/or ONO₂;
   wherein at least one of said R₂₂, R₂₃ and R₂₄ comprises at least one ONO₂ moiety.

In a preferred embodiment, said compound of formula I is a compound of formula I*, or pharmaceutically acceptable salt, solvate or hydrate thereof,

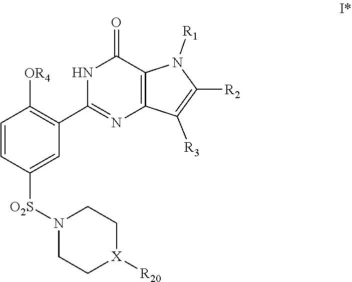

wherein R₁, R₂, R₃, and R₄, are as defined herein including all preferred and very preferred embodiments of R₁, R₂, R₃, and R₄ alone or in combination, and wherein
X is CR₂₁ or N;
R₂₀ is C₁-C₆alkyl optionally substituted with OH and/or ONO₂;
R₂₁ is H or C₁-C₆alkyl optionally substituted with OH and/or ONO₂;
   wherein at least one of said R₂₀ and R₂₁ comprises at least one ONO₂ moiety.

In a further preferred embodiment, said R₂₀ is C₁-C₆alkyl optionally substituted with OH and/or ONO₂; said R₂₁ is H or C₁-C₄alkyl optionally substituted with OH and/or ONO₂; wherein at least one of said R₂₀ and R₂₁ comprises at least one ONO₂ moiety. In a further preferred embodiment, said R₂₀ is C₁-C₆alkyl optionally substituted with OH and/or ONO₂; said R₂₁ is H or C₁-C₃alkyl optionally substituted with OH and/or ONO₂; wherein at least one of said R₂₀ and R₂₁ comprises at least one ONO₂ moiety. In a further preferred embodiment, said R₂₀ is C₁-C₆alkyl optionally substituted with OH and/or ONO₂; said R₂₁ is H or C₁-C₂alkyl optionally substituted with OH and/or ONO₂; wherein at least one of said R₂₀ and R₂₁ comprises at least one ONO₂ moiety. In a further preferred embodiment, said X is CH or N; said R₂₀ is C₁-C₆alkyl optionally substituted with OH and/or ONO₂. In a further preferred embodiment, said R₂₀ is C₁-C₄alkyl optionally substituted with OH and/or ONO₂; said R₂₁ is H or C₁-C₄alkyl optionally substituted with OH and/or ONO₂; wherein at least one of said R₂₀ and R₂₁ comprises at least one ONO₂ moiety. In a further preferred embodiment, said R₂₀ is C₁-C₃alkyl optionally substituted with OH and/or ONO₂; said R₂₁ is H or C₁-C₃alkyl optionally substituted with OH and/or ONO₂; wherein at least one of said R₂₀ and R₂₁ comprises at least one ONO₂ moiety. In a further preferred embodiment, said R₂₀ is C₁-C₄alkyl optionally substituted with OH and/or ONO₂; said R₂₁ is H or C₁-C₂alkyl optionally substituted with OH and/or ONO₂; wherein at least one of said R₂₀ and R₂₁ comprises at least one ONO₂ moiety.

In a further preferred embodiment, said R₂₀ or R₂₁ independently of each other comprises at least one ONO₂ moiety. In a further preferred embodiment, said R₂₀ or said R₂₁ comprises exactly one ONO₂ moiety. In a further very preferred embodiment, said R₂₀ comprises exactly one ONO₂ moiety. In a further very preferred embodiment, said R₂₁ comprises exactly one ONO₂ moiety. In a further preferred embodiment, said R₂₀ and said R₂₁ comprise together at least two ONO₂ moieties. In a further preferred embodiment, said R₂₀ and said R₂₁ comprise together exactly two ONO₂ moieties. In a further preferred embodiment, said R₂₀ comprises exactly two ONO₂ moieties. In a further preferred embodiment, said R₂₀ and said R₂₁ each comprises exactly one ONO₂ moiety. In a further preferred embodiment, said R₂₀ and said R₂₁ each comprises exactly one ONO₂ moiety. In a further preferred embodiment, said R₂₀ and said R₂₁ comprise together at least three ONO₂ moieties. In a further preferred embodiment, said R₂₀ and said R₂₁ comprise together exactly three ONO₂ moieties. In a preferred embodiment, said R₂₁ is H.

In a further preferred embodiment, said R₂₀ is C₁-C₃alkyl optionally substituted with OH and/or ONO₂; said R₂₁ is H or C₁-C₃alkyl optionally substituted with OH and/or ONO₂; wherein each of said R₂₀ and R₂₁ independently of each other comprises at least one, preferably one or two, ONO₂ moiety. In a further preferred embodiment, said R₂₀ is C₁-C₄alkyl optionally substituted with OH and/or ONO₂; said R₂₁ is H or C₁-C₂alkyl optionally substituted with OH and/or ONO₂; wherein each of said R₂₀ and R₂₁ independently of each other comprises at least one, preferably one or two, ONO₂ moiety. In a further preferred embodiment, said R₂₀ is C₁-C₃alkyl optionally substituted with OH and/or ONO₂; said R₂₁ is H or C₁-C₃alkyl optionally substituted with OH and/or ONO₂; wherein each of said R₂₀ and R₂₁ comprises exactly one ONO₂ moiety. In a further preferred embodiment, said R₂₀ is C₁-C₄alkyl optionally substituted with OH and/or ONO₂; said R₂₁ is H or C₁-C₂alkyl optionally substituted with OH and/or ONO₂; wherein each of said R₂₀ and R₂₁ comprises exactly one ONO₂ moiety. In a further preferred embodiment, said R₂₀ is C₂-C₃alkyl optionally substituted with OH and/or ONO₂; said R₂₁ is H or C₂-C₃alkyl optionally substituted with OH and/or ONO₂; wherein each of said R₂₀ and R₂₁ independently of each other comprises at least one, preferably one or two, ONO₂ moiety. In a further preferred embodiment, said R₂₀ is C₂-C₃alkyl optionally substituted with OH and/or ONO₂; said R₂₁ is H or C₂-C₃alkyl optionally substituted with OH and/or ONO₂; wherein each of said R₂₀ and R₂₁ comprises exactly one ONO₂ moiety. In a further preferred embodiment, said R₂₀ is selected from CH₂ONO₂, CH₂CH₂ONO₂, CH(OH)CH₂ONO₂, CH₂CH₂CH₂ONO₂, CH(ONO₂)CH₂OH, CH(ONO₂)CH₂ONO₂, C(OH)(CH₂ONO₂)CH₂ONO₂, C(OH)(CH₂CH₂ONO₂)CH₂CH₂ONO₂, and wherein said R₂₁ is selected from H, CH₂ONO₂, CH₂CH₂ONO₂, CH(OH)CH₂ONO₂, CH₂CH₂CH₂ONO₂, CH(ONO₂)CH₂OH, CH(ONO₂)CH₂ONO₂. In a further very preferred embodiment, said R₂₀ and said R₂₁ is CH₂CH₂ONO₂.

In a further preferred embodiment, said compound of formula I is a compound of formula I**, or pharmaceutically acceptable salt, solvate or hydrate thereof,

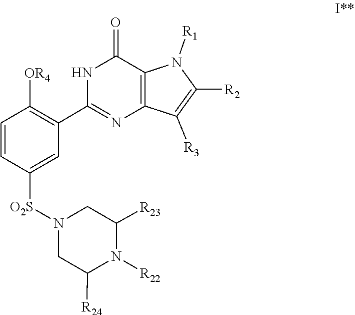

wherein R₁, R₂, R₃, and R₄, are as defined herein including all preferred and very preferred embodiments of R₁, R₂, R₃, and R₄ alone or in combination, and wherein
R₂₂ is H or C₁-C₆alkyl optionally substituted with OH and/or ONO₂;
R₂₃ and R₂₄ are each independently C₁-C₆alkyl optionally substituted with OH and/or ONO₂;
wherein at least one of said R₂₂, R₂₃ and R₂₄ comprises at least one ONO₂ moiety.

In a further preferred embodiment, said R₂₂ is H or C₁-C₄alkyl optionally substituted with OH and/or ONO₂; said R₂₃ and R₂₄ are each independently C₁-C₄alkyl optionally substituted with OH and/or ONO₂. In a further preferred embodiment, said R₂₂ is H or C₁-C₂alkyl, preferably H or methyl; said R₂₃ and R₂₄ are each independently C₁-C₃alkyl optionally substituted with OH and/or ONO₂. In a further preferred embodiment, said R₂₂ is H or methyl; said R₂₃ and R₂₄ are each independently C₁-C₂alkyl optionally substituted with OH and/or ONO₂. In a further preferred embodiment, said R₂₂ is H or methyl; said R₂₃ and R₂₄ are each independently C₂-C₃alkyl optionally substituted with OH and/or ONO₂. In a further preferred embodiment, said R₂₂ is methyl; said R₂₃ and R₂₄ are each independently C₁-C₂alkyl optionally substituted with OH and/or ONO₂. In a further preferred embodiment, said R₂₂ is methyl; said R₂₃ and R₂₄ are each independently C₂-C₃alkyl optionally substituted with OH and/or ONO₂.

In a further preferred embodiment, said R₂₂, R₂₃ and R₂₄ comprise together at least two ONO₂ moieties. In a further preferred embodiment, said R₂₂, R₂₃ and R₂₄ comprise together exactly two ONO₂ moieties. In a further preferred embodiment, said R₂₃ and said R₂₄ comprise together exactly two ONO₂ moieties. In a further preferred embodiment, said R₂₃ and said R₂₄ comprise exactly one ONO₂ moiety.

In a further preferred embodiment, said compound of formula I is a compound selected from

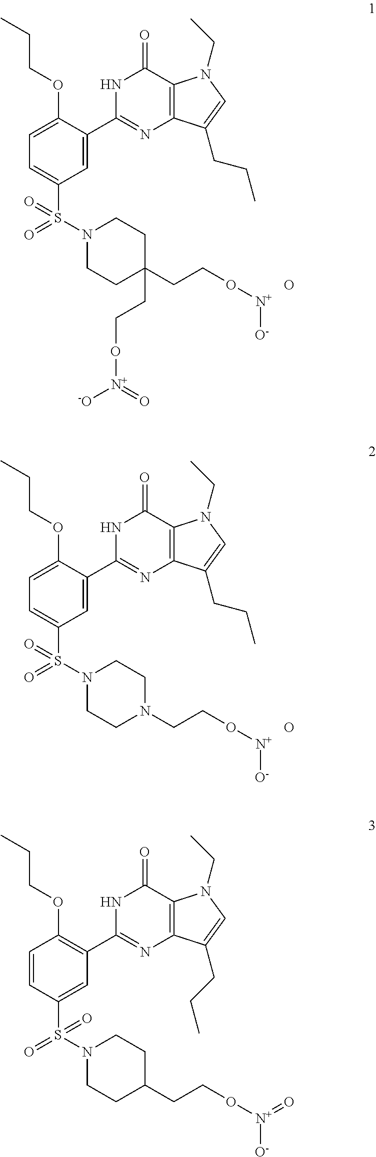

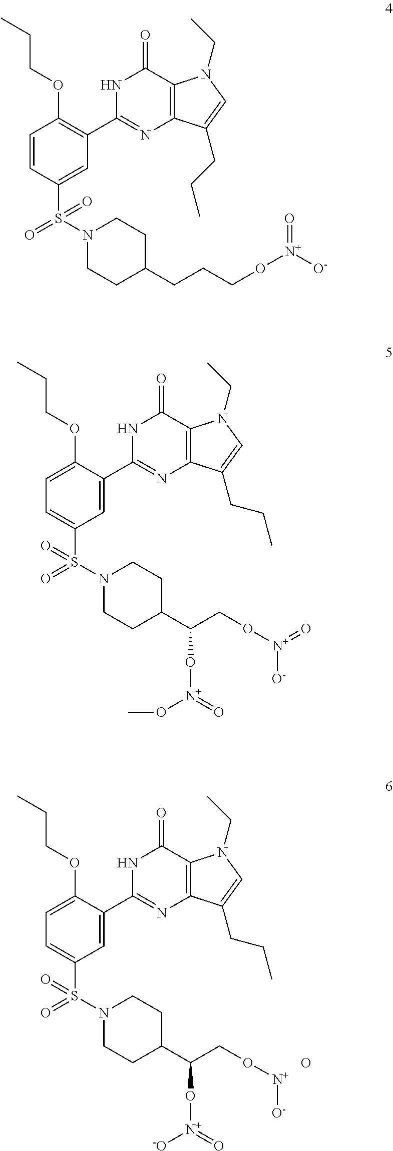

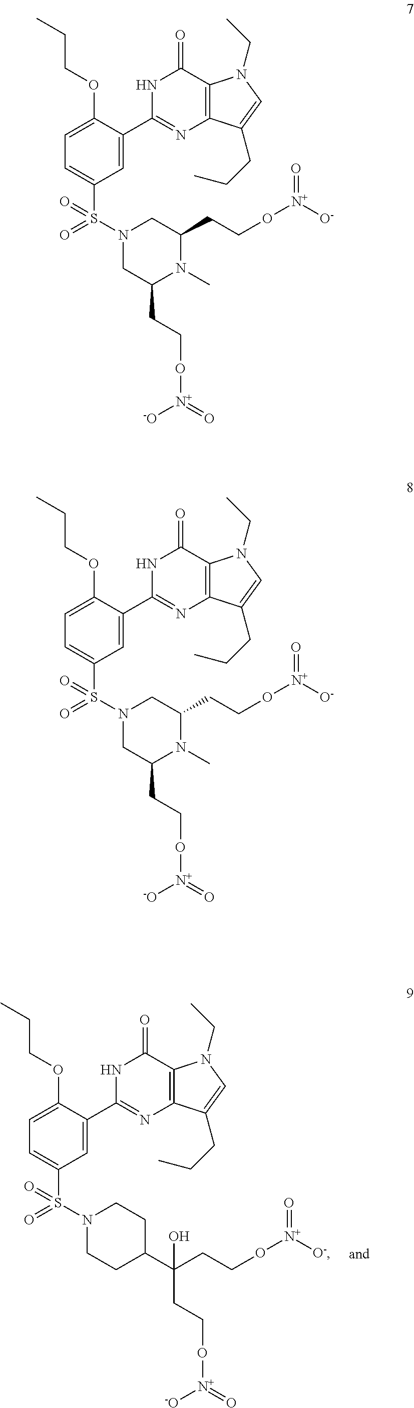

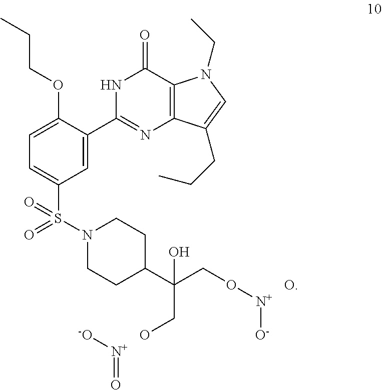

In a further preferred embodiment, said compound of formula I is a compound selected from
(1-((3-(5-ethyl-4-oxo-7-propyl-4,5-dihydro-3H-pyrrolo[3,2-d]pyrimidin-2-yl)-4-propoxyphenyl)sulfonyl)piperidine-4,4-diyl)bis(ethane-2,1-diyl) dinitrate (1);
2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl nitrate (2);
2-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethyl nitrate (3);
3-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)propyl nitrate (4);
(R)-1-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethane-1,2-diyl dinitrate (5);
(S)-1-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethane-1,2-diyl dinitrate (6);
((2R,6S)-4-((3-(5-ethyl-4-oxo-7-propyl-4,5-dihydro-3H-pyrrolo[3,2-d]pyrimidin-2-yl)-4-propoxyphenyl)sulfonyl)-1-methylpiperazine-2,6-diyl)bis(ethane-2,1-diyl) dinitrate (7);
((2S,6S)-4-((3-(5-ethyl-4-oxo-7-propyl-4,5-dihydro-3H-pyrrolo[3,2-d]pyrimidin-2-yl)-4-propoxyphenyl)sulfonyl)-1-methylpiperazine-2,6-diyl)bis(ethane-2,1-diyl) dinitrate (8);
3-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)-3-hydroxypentane-1,5-diyl dinitrate (9); and
2-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)-2-hydroxypropane-1,3-diyl dinitrate (10).

In a further preferred embodiment, said compound of formula I is Compound 1 or Compound 2

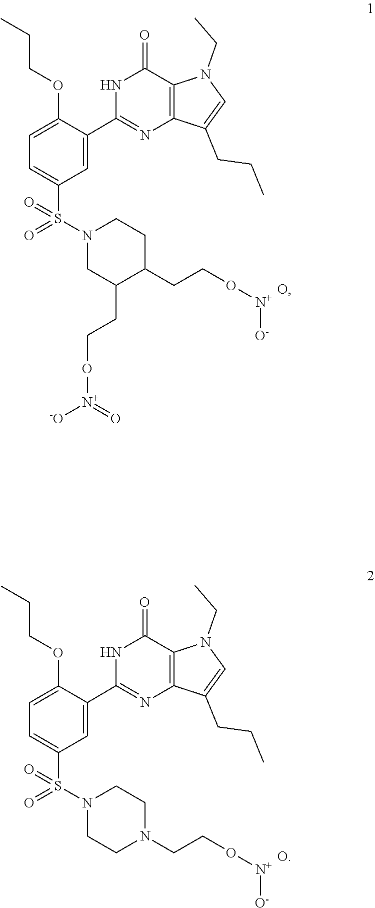

In a further very preferred embodiment, said compound of formula I is Compound 1

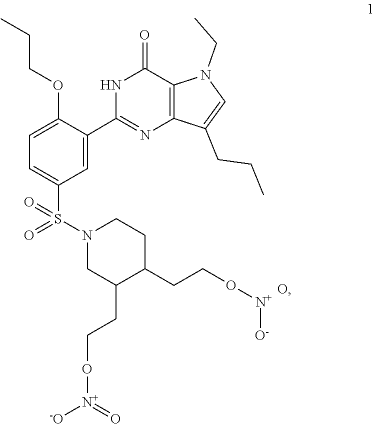

In a preferred embodiment, said compound of formula I, preferably said Compound 1, is present in said topical composition from about 0.0001% (w/v) to about 0.02% (w/v) based on the total volume of said topical composition. In a preferred embodiment, said compound of formula I, preferably said Compound 1, is present in said topical composition from about 0.0002% (w/v) to about 0.02% (w/v) based on the total volume of said topical composition. In a preferred embodiment, said compound of formula I, preferably said Compound 1, is present in said topical composition from about 0.0002% (w/v) to about 0.01% (w/v) based on the total volume of said topical composition. In a preferred embodiment, said topical composition comprises said compound of formula I, preferably said Compound 1, from about 0.0002% (w/v) to about 0.02% (w/v). In a preferred embodiment, said topical composition comprises said compound of formula I, preferably said Compound 1, from about 0.0002% (w/v) to about 0.01% (w/v).

In a preferred embodiment, said compound of formula I, preferably said Compound 1, is present in said topical composition from about 0.0002% (w/v) to about 0.008% (w/v) based on the total volume of said topical composition. In a preferred embodiment, said compound of formula I, preferably said Compound 1, is present in said topical composition from about 0.0002% (w/v) to about 0.006% (w/v) based on the total volume of said topical composition.

In a preferred embodiment, said topical composition comprises said compound of formula I, preferably said Compound 1, from about 0.0002% (w/v) to about 0.008% (w/v). In a preferred embodiment, said topical composition comprises said compound of formula I, preferably said Compound 1, from about 0.0002% (w/v) to about 0.006% (w/v).

In a preferred embodiment, said compound of formula I, preferably said Compound 1, is present in said topical composition from about 0.0002% (w/v) to about 0.005% (w/v) based on the total volume of said topical composition. In a preferred embodiment, said compound of formula I, preferably said Compound 1, is present in said topical composition from about 0.0002% (w/v) to about 0.004% (w/v) based on the total volume of said topical composition.

In a preferred embodiment, said topical composition comprises said compound of formula I, preferably said Compound 1, from about 0.0002% (w/v) to about 0.005% (w/v). In a preferred embodiment, said topical composition comprises said compound of formula I, preferably said Compound 1, from about 0.0002% (w/v) to about 0.004% (w/v).

In a preferred embodiment, said compound of formula I is Compound 1, and said Compound 1 is present in said topical composition from about 0.0001% (w/v) to about 0.02% (w/v) based on the total volume of said topical composition. In a preferred embodiment, said compound of formula I is Compound 1, and said Compound 1 is present in said topical composition from about 0.0002% (w/v) to about 0.02% (w/v) based on the total volume of said topical composition. In a preferred embodiment, said compound of formula I is Compound 1, and said Compound 1 is present in said topical composition from about 0.0002% (w/v) to about 0.01% (w/v) based on the total volume of said topical composition. In a preferred embodiment, said compound of formula I is Compound 1, and said topical composition comprises said Compound 1 from about 0.0002% (w/v) to about 0.02% (w/v). In a preferred embodiment, said compound of formula I is Compound 1, and said topical composition comprises said Compound 1 from about 0.0002% (w/v) to about 0.01% (w/v).

In a preferred embodiment, said compound of formula I is Compound 1, and said Compound 1 is present in said topical composition from about 0.0002% (w/v) to about 0.008% (w/v) based on the total volume of said topical composition. In a preferred embodiment, said compound of formula I is Compound 1, and said Compound 1 is present in said topical composition from about 0.0002% (w/v) to about 0.006% (w/v) based on the total volume of said topical composition. In a preferred embodiment, said compound of formula I is Compound 1, and said topical composition comprises said Compound 1 from about 0.0002% (w/v) to about 0.008% (w/v). In a preferred embodiment, said compound of formula I is Compound 1, and said topical composition comprises said Compound 1 from about 0.0002% (w/v) to about 0.006% (w/v).

In a preferred embodiment, said compound of formula I is Compound 1, and said Compound 1 is present in said topical composition from about 0.0002% (w/v) to about 0.005% (w/v) based on the total volume of said topical composition. In a preferred embodiment, said compound of formula I is Compound 1, and said Compound 1 is present in said topical composition from about 0.0002% (w/v) to about 0.004% (w/v) based on the total volume of said topical composition. In a preferred embodiment, said compound of formula I is Compound 1, and said topical composition comprises said Compound 1 from about 0.0002% (w/v) to about 0.005% (w/v). In a preferred embodiment, said compound of formula I is Compound 1, and said topical composition comprises said Compound 1 from about 0.0002% (w/v) to about 0.004% (w/v).

In a further preferred embodiment, said topical composition comprises said compound of formula I, preferably said Compound 1, in a concentration between about 1.5 μM and about 300 μM, preferably between about 3 μM and about 200 μM, further preferably between about 5 μM and about 100 μM.

In a further preferred embodiment, said compound of formula I is Compound 1, and said topical composition comprises said Compound 1 in a concentration between about 1.5 μM and about 300 μM, preferably between about 3 μM and about 200 μM, further preferably between about 5 μM and about 100 μM.

In a further preferred embodiment, said topical composition comprises said compound of formula I, preferably said Compound 1, in a concentration between about 5 μM, preferably about 6 μM, and about 75 μM. In a further preferred embodiment, said topical composition comprises said compound of formula I, preferably said Compound 1, in a concentration between about 5 μM, preferably about 6 μM, and about 60 μM. In a further preferred embodiment, said topical composition comprises said compound of formula I, preferably said Compound 1, in a concentration between about 5 μM, preferably about 6 μM, and about 50 μM. In a further preferred embodiment, said topical composition comprises said compound of formula I, preferably said Compound 1, in a concentration between about 5 μM, preferably about 6 μM, and about 45 μM. In a further preferred embodiment, said topical composition comprises said compound of formula I, preferably said Compound 1, in a concentration between about 5 μM, preferably about 6 μM, and about 40 μM. In a further preferred embodiment, said topical composition comprises said compound of formula I, preferably said Compound 1, in a concentration between about 6 μM, and about 40 μM. In a further preferred embodiment, said topical composition comprises said compound of formula I, preferably said Compound 1, in a concentration between about 6 μM and about 38 μM. In a further preferred embodiment, said topical composition comprises said compound of formula I, preferably said Compound 1, in a concentration between about 6 μM and about 35 μM. In a further preferred embodiment, said topical composition comprises said compound of formula I, preferably said Compound 1, in a concentration between about 6 μM and about 8 μM. In a further preferred embodiment, said topical composition comprises said compound of formula I, preferably said Compound 1, in a concentration between about 25 μM and about 35 μM. In a further preferred embodiment, said topical composition comprises said compound of formula I, preferably said Compound 1, in a concentration of about 6 μM. In a further preferred embodiment, said topical composition comprises said compound of formula I, preferably said Compound 1, in a concentration of about 30 μM.

In a further preferred embodiment, said compound of formula I is Compound 1, and said topical composition comprises said Compound 1 in a concentration between about 5 μM, preferably about 6 μM, and about 75 μM. In a further preferred embodiment, said compound of formula I is Compound 1, and said topical composition comprises said Compound 1 in a concentration between about 5 μM, preferably about 6 μM, and about 60 μM. In a further preferred embodiment, said compound of formula I is Compound 1, and said topical composition comprises said Compound 1 in a concentration between about 5 μM, preferably about 6 μM, and about 50 μM. In a further preferred embodiment, said compound of formula I is Compound 1, and said topical composition comprises said Compound 1 in a concentration between about 5 μM, preferably about 6 μM, and about 45 μM. In a further preferred embodiment, said compound of formula I is Compound 1, and said topical composition comprises said Compound 1 in a concentration between about 5 μM, preferably about 6 μM, and about 40 μM. In a further preferred embodiment, said compound of formula I is Compound 1, and said topical composition comprises said Compound 1 in a concentration between about 6 μM, and about 40 μM. In a further preferred embodiment, said compound of formula I is Compound 1, and said topical composition comprises said Compound 1 in a concentration between about 6 μM and about 38 μM. In a further preferred embodiment, said compound of formula I is Compound 1, and said topical composition comprises said Compound 1 in a concentration between about 6 μM and about 35 μM. In a further preferred embodiment, said compound of formula I is Compound 1, and said topical composition comprises said Compound 1 in a concentration between about 6 μM and about 8 μM. In a further preferred embodiment, said compound of formula I is Compound 1, and said topical composition comprises said Compound 1 in a concentration between about 25 μM and about 35 μM. In a further preferred embodiment, said compound of formula I is Compound 1, and said topical composition comprises said Compound 1 in a concentration of about 6 μM. In a further preferred embodiment, said compound of formula I is Compound 1, and said topical composition comprises said Compound 1 in a concentration of about 30 μM.

In some embodiments, said topical composition can be formulated to different dosage forms, for example, a solution, a suspension, a cream, an ointment, a lotion, a paste, an emulsion, a foam and a gel.

In a preferred embodiment, said topical composition is formulated as a solution or a gel. In a preferred embodiment, said topical composition is a liquid topical composition. In a further preferred embodiment, said topical composition is a liquid topical composition, wherein said liquid topical composition is formulated as a solution. In a further preferred embodiment, said topical composition is a liquid topical composition, wherein said liquid topical composition is formulated as a gel.

In a preferred embodiment, said topical composition comprises at least one solvent, wherein said solvent is selected from the group consisting of PEG 200, PEG 240, PEG 300, PEG 350, PEG 400, PEG 540, ethanol, 2-(2-ethoxyethoxy)ethanol (transcutol), glycerin, propylene glycol, polypropylene glycol, poloxamer 101, poloxamer 182, poloxamer 188, poloxamer 237, poloxamer 331, poloxamer 338, poloxamer 407, water, and mixtures thereof. In a preferred embodiment, said topical composition comprises at least one solvent, wherein said at least one solvent is selected from the group consisting of PEG 400, PEG 350, PEG 240, ethanol, 2-(2-ethoxyethoxy)ethanol (transcutol), glycerin, propylene glycol polypropylene glycol, poloxamer 407, poloxamer 188 and water. As used herein, the term “transcutol” refers to 2-(2-ethoxyethoxy)ethanol.

In a preferred embodiment, said topical composition comprises a combination of solvents, wherein said combination of solvents comprises solvents selected from the group consisting of PEG 200, PEG 240, PEG 300, PEG 350, PEG 400, PEG 540, ethanol, 2-(2-ethoxyethoxy)ethanol (transcutol), glycerin, propylene glycol, polypropylene glycol, poloxamer 101, poloxamer 182, poloxamer 188, poloxamer 237, poloxamer 331, poloxamer 338, poloxamer 407, and water. In a preferred embodiment, said topical composition comprises a combination of solvents, wherein said combination of solvents comprises solvents selected from the group consisting of PEG 400, PEG 350, PEG 240, ethanol, 2-(2-ethoxyethoxy)ethanol (transcutol), glycerin, propylene glycol polypropylene glycol, poloxamer 407, poloxamer 188 and water.

In a preferred embodiment, said topical composition comprises a combination of solvents, wherein said combination of solvents comprises PEG 400 and ethanol. In a preferred embodiment, said topical composition comprises a combination of solvents, wherein said combination of solvents comprises, preferably consists of, PEG 400 and ethanol, and wherein the amount of said PEG 400 is from about 60% to about 75% (vol/vol), preferably from about 65% to about 75% (vol/vol)), and more preferably from about 68% to about 72% (vol/vol) based on the total volume of the topical composition, and wherein the amount of said ethanol is from about 25% to about 40% (vol/vol), preferably from about 25% to about 35% (vol/vol)), and more preferably from about 28% to about 32% (vol/vol) based on the total volume of the topical composition.

In a preferred embodiment, said topical composition comprises a combination of solvents, wherein said combination of solvents consists of PEG 400 and ethanol, and wherein the amount of said PEG 400 is from about 60% to about 75% (vol/vol), preferably from about 65% to about 75% (vol/vol)), and more preferably from about 68% to about 72% (vol/vol) based on the total volume of the topical composition, and wherein the amount of said ethanol is from about 25% to about 40% (vol/vol), preferably from about 25% to about 35% (vol/vol)), and more preferably from about 28% to about 32% (vol/vol) based on the total volume of the topical composition.

In a preferred embodiment, said topical composition comprises a combination of solvents, wherein said combination of solvents consists of PEG 400 and ethanol, and wherein the amount of said PEG 400 is from about 68% to about 72% (vol/vol) based on the total volume of the topical composition, and wherein the amount of said ethanol is from about 28% to about 32% (vol/vol) based on the total volume of the topical composition.

In a preferred embodiment, said topical composition comprises a combination of solvents, wherein said combination of solvents consists of PEG 400 and ethanol, and wherein the amount of said PEG 400 is about 70% (vol/vol) based on the total volume of the topical composition, and wherein the amount of said ethanol is about 30% (vol/vol) based on the total volume of the topical composition.

In a preferred embodiment, said topical composition comprises a solvent, wherein said solvent is a combination of PEG 400 and ethanol, wherein the ratio (vol/vol) of said PEG 400 and ethanol is from about 60:40 to 75:25, preferably from about 65:35 to 75:25, further preferably from about 68:32 to 72:28, and again further preferably about 70:30.

In a preferred embodiment, said topical composition comprises a combination of solvents, wherein said combination of solvents comprises, preferably consists of, PEG 400, ethanol, transcutol and water, and wherein the amount of said PEG 400 is from about 5% to about 15% (vol/vol), preferably from about 8% to about 12% (vol/vol)), based on the total volume of the topical composition, and wherein the amount of said ethanol is from about 20% to about 40% (vol/vol), preferably from about 25% to about 35% (vol/vol)) based on the total volume of the topical composition, and wherein the amount of said transcutol is from about 5% to about 15% (vol/vol), preferably from about 8% to about 12% (vol/vol)), based on the total volume of the topical composition, and wherein the amount of said water is from about 30% to about 70% (vol/vol), preferably from about 40% to about 60%, further preferably from about 45% to about 55% (vol/vol)) based on the total volume of the topical composition.

In a preferred embodiment, said topical composition comprises a combination of solvents, wherein said combination of solvents consists of PEG 400, ethanol, transcutol and water, and wherein the amount of said PEG 400 is from about 5% to about 15% (vol/vol), preferably from about 8% to about 12% (vol/vol)), based on the total volume of the topical composition, and wherein the amount of said ethanol is from about 20% to about 40% (vol/vol), preferably from about 25% to about 35% (vol/vol)) based on the total volume of the topical composition, and wherein the amount of said transcutol is from about 5% to about 15% (vol/vol), preferably from about 8% to about 12% (vol/vol)), based on the total volume of the topical composition, and wherein the amount of said water is from about 30% to about 70% (vol/vol), preferably from about 40% to about 60%, further preferably from about 45% to about 55% (vol/vol)) based on the total volume of the topical composition.

In a preferred embodiment, said topical composition comprises a solvent, wherein said solvent is a combination of PEG 400, ethanol, transcutol and water, wherein the ratio (vol/vol) of said PEG400:ethanol:transcutol:water is from about 5:20:5:30 to 15:40:15:70, preferably from about 8:25:8:40 to about 12:35:12:60, and further preferably about 10:30:10:50.

In a preferred embodiment, the solvent is present at a concentration from about 90% w/w to about 99% w/w, preferably about 95% w/w to about 99% w/w, more preferably about 97% w/w to about 98% w/w, based on the total weight of the topical composition.

In a preferred embodiment, said topical composition comprises optionally one or more other pharmaceutically acceptable excipients. In a preferred embodiment, said topical composition comprises one or more other pharmaceutically acceptable excipients. In a preferred embodiment, said pharmaceutically acceptable excipient is selected from the group consisting of a thickening agent, a stabilizer, an antioxidant, a chelating agent, an oily material, an emulsifier, a penetration enhancer, a pH adjusting agent, a preservative, an antimicrobial agent, an opacifier, a fragrance, a colorant, a gelling agent, a moisturizer, a surfactant, and a combination thereof. In one embodiment, said pharmaceutically acceptable excipient is selected from the group consisting of a thickening agent, a stabilizer, an antioxidant, a pH adjusting agent, a preservative, an antimicrobial agent, and a combination thereof.

The pharmaceutically acceptable excipients used in the topical composition of the present invention can act in more than one way. For example, a thickening agent can also function as a gelling agent and a solubilizing agent can also function as a solvent.

In a preferred embodiment, said topical composition comprises one or more other pharmaceutically acceptable excipients, and one of said pharmaceutically acceptable excipients is a thickening agent. In a preferred embodiment, said topical composition comprises a thickening agent. Thickening agents are known to the skilled person in the art and includes a cellulose derivative, polyvinylpyrrolidone, a carbomer polymer, carbomer derivative, maltodextrin, polydextrose, dextrates, carboxypolymethylene, polyvinyl alcohol, poloxamers and mixtures thereof.

In a preferred embodiment, said topical composition comprises one or more pharmaceutically acceptable excipient selected from a cellulose derivative, polyvinylpyrrolidone, a carbomer polymer, carbomer derivative, maltodextrin, polydextrose, dextrates, carboxypolymethylene, polyvinyl alcohol, poloxamers or a mixture thereof.

In a preferred embodiment, said topical composition comprises one or more other pharmaceutically acceptable excipients, and one of said pharmaceutically acceptable excipients is an antioxidant. In a preferred embodiment, said topical composition comprises an antioxidant. Antioxidants are known to the skilled person in the art and includes, for example, butylated hydroxyanisole, butylated hydroxytoluene, vitamin C, vitamin E, vitamin A, lutein, lycopene, retinyl palmitate, potassium metabisulfite, sodium metabisulfite, sodium thiosulfate pentahydrate, 3,4-dihydroxybenzoic acid, propyl gallate, alpha-lipoic acid, ascorbyl palmitate, sodium pyrosulfite, ubiquinone, selenium, or a combination thereof.

In a preferred embodiment, said topical composition comprises one or more pharmaceutically acceptable excipient selected from butylated hydroxyanisole, butylated hydroxytoluene, vitamin C, vitamin E, vitamin A, lutein, lycopene, retinyl palmitate, potassium metabisulfite, sodium metabisulfite, sodium thiosulfate pentahydrate, 3,4-dihydroxybenzoic acid, propyl gallate, alpha-lipoic acid, ascorbyl palmitate, sodium pyrosulfite, ubiquinone, selenium, or a combination thereof.

In a preferred embodiment, said topical composition comprises one or more other pharmaceutically acceptable excipients, and one of said pharmaceutically acceptable excipients is a preservative. In a preferred embodiment, said topical composition comprises a preservative.

Preservatives are known to the skilled person in the art and includes, for example, benzyl alcohol, benzoic acid, phenol, m-cresol, methyl parabene, propyl parabene, or a combination thereof.

In a preferred embodiment, said topical composition comprises one or more pharmaceutically acceptable excipient selected from benzyl alcohol, benzoic acid, phenol, m-cresol, methyl parabene, propyl parabene, or a combination thereof.

In a preferred embodiment, said topical composition comprises one or more pharmaceutically acceptable excipient selected PEG-20 hydrogenated castor oil, PEG-35 hydrogenated castor oil, PEG-40 hydrogenated castor oil (polyoxyl 40 hydrogenated castor oil), PEG-60 hydrogenated castor oil, PEG-100 hydrogenated castor oil, PEG-1000 hydrogenated castor oil, PEG-2000 hydrogenated castor oil, PEG-4000 hydrogenated castor oil, PEG-6000 hydrogenated castor oil, polysorbate 20, polysorbate 40, polysorbate 60, polysorbate 80, and polysorbate 100.

In a preferred embodiment, said topical composition comprises one or more pharmaceutically acceptable excipient selected PEG-20 hydrogenated castor oil, PEG-35 hydrogenated castor oil, PEG-40 hydrogenated castor oil (polyoxyl 40 hydrogenated castor oil), PEG-60 hydrogenated castor oil, PEG-100 hydrogenated castor oil, PEG-1000 hydrogenated castor oil, PEG-2000 hydrogenated castor oil, PEG-4000 hydrogenated castor oil, PEG-6000 hydrogenated castor oil, polysorbate 20, polysorbate 40, polysorbate 60, polysorbate 80, polysorbate 100, cellulose derivative, polyvinylpyrrolidone, a carbomer polymer, carbomer derivative, maltodextrin, polydextrose, dextrates, carboxypolymethylene, polyvinyl alcohol, poloxamers, butylated hydroxyanisole, butylated hydroxytoluene, vitamin C, vitamin E, vitamin A, lutein, lycopene, retinyl palmitate, potassium metabisulfite, sodium metabisulfite, sodium thiosulfate pentahydrate, 3,4-dihydroxybenzoic acid, propyl gallate, alpha-lipoic acid, ascorbyl palmitate, sodium pyrosulfite, ubiquinone, selenium, benzyl alcohol, benzoic acid, phenol, m-cresol, methyl parabene, propyl parabene, or a combination or a mixture thereof.

In a preferred embodiment, said compound of formula I is Compound 1,

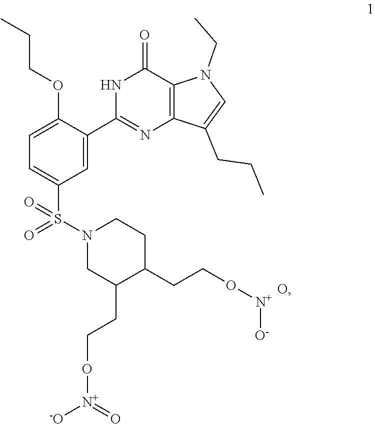

and
wherein said topical composition comprises said Compound 1 in a concentration between about 1.5 μM and about 300 μM, preferably between about 3 μM and about 200 μM, further preferably between about 5 μM and about 100 μM; and
wherein said topical composition comprises a combination of solvents, wherein said combination of solvents comprises, preferably consists of, PEG 400 and ethanol, and wherein the amount of said PEG 400 is from about 60% to about 75% (vol/vol), preferably from about 65% to about 75% (vol/vol)), and more preferably from about 68% to about 72% (vol/vol) based on the total volume of the topical composition, and wherein the amount of said ethanol is from about 25% to about 40% (vol/vol), preferably from about 25% to about 35% (vol/vol)), and more preferably from about 28% to about 32% (vol/vol) based on the total volume of the topical composition.

In a preferred embodiment, said compound of formula I is Compound 1,

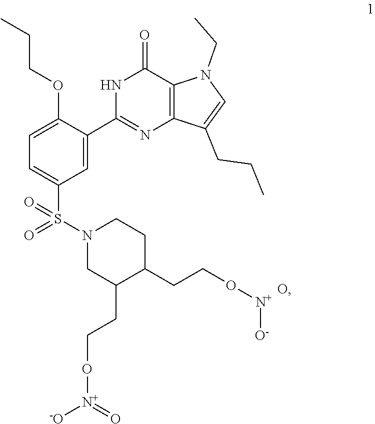

and
wherein said topical composition comprises said Compound 1 in a concentration between about 1.5 μM and about 300 μM, preferably between about 3 μM and about 200 μM, further preferably between about 5 μM and about 100 μM; and
wherein said topical composition comprises a combination of solvents, wherein said combination of solvents comprises, preferably consists of, PEG 400, ethanol, transcutol and water, and wherein the amount of said PEG 400 is from about 5% to about 15% (vol/vol), preferably from about 8% to about 12% (vol/vol)), based on the total volume of the topical composition, and wherein the amount of said ethanol is from about 20% to about 40% (vol/vol), preferably from about 25% to about 35% (vol/vol)) based on the total volume of the topical composition, and wherein the amount of said transcutol is from about 5% to about 15% (vol/vol), preferably from about 8% to about 12% (vol/vol)), based on the total volume of the topical composition, and wherein the amount of said water is from about 30% to about 70% (vol/vol), preferably from about 40% to about 60%, further preferably from about 45% to about 55% (vol/vol)) based on the total volume of the topical composition.

In a preferred embodiment, said compound of formula I is Compound 1,

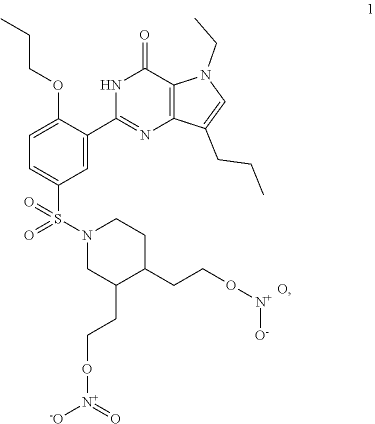

and
wherein said topical composition comprises said Compound 1 in a concentration between about 5 μM and about 75 μM, preferably between about 5 μM and about 60 μM, further preferably between about 5 μM and about 50 μM; and
wherein said topical composition comprises a combination of solvents, wherein said combination of solvents consists of PEG 400 and ethanol, and wherein the amount of said PEG 400 is from about 68% to about 72% (vol/vol) based on the total volume of the topical composition, and wherein the amount of said ethanol is from about 28% to about 32% (vol/vol) based on the total volume of the topical composition.

In a preferred embodiment, said compound of formula I is Compound 1,

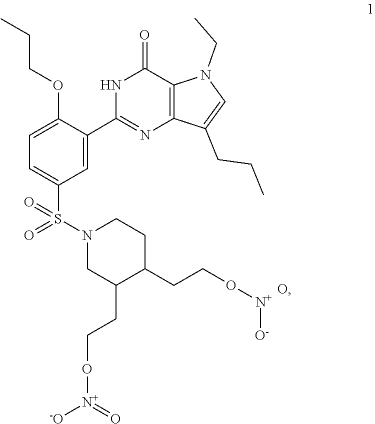

and
wherein said topical composition comprises said Compound 1 in a concentration between about 1.5 μM and about 300 μM, preferably between about 3 μM and about 200 μM, further preferably between about 5 μM and about 100 μM; and
wherein said topical composition comprises a solvent, wherein said solvent is a combination of PEG 400, ethanol, transcutol and water, wherein the ratio (vol/vol) of said PEG400:ethanol:transcutol:water is from about 5:20:5:30 to 15:40:15:70, preferably from about 8:25:8:40 to about 12:35:12:60, and further preferably about 10:30:10:50

In a preferred embodiment, said compound of formula I is Compound 1,

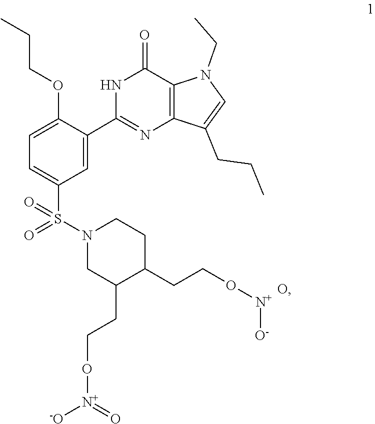

and
wherein said topical composition comprises said Compound 1 in a concentration between about 5 μM and about 40 μM, preferably between about 5 μM and about 38 μM, further preferably between about 5 μM and about 35 μM; and
wherein said topical composition comprises a solvent, wherein said solvent is a combination of PEG 400 and ethanol, wherein the ratio (vol/vol) of said PEG 400 and ethanol is from about 60:40 to 75:25, preferably from about 65:35 to 75:25, further preferably from about 68:32 to 72:28, and again further preferably about 70:30.

The topical composition of the present invention can be used for the topical treatment of diseases or disorders of the skin being mediated by or resulting from PDE5 activity and/or NO related endothelial dysfunction. Moreover, the topical composition of the present invention can be used for the topical treatment of diseases or disorders being mediated by or resulting from PDE5 activity and/or NO related endothelial dysfunction. Furthermore, the topical composition of the present invention can be used for the topical treatment of diseases or disorders of the skin being mediated by or resulting from PDE5 activity and/or NO related endothelial dysfunction, In particular, the topical composition of the present invention can be used for the topical treatment of diseases or disorders in a subject, preferably in a human, wherein said topically treating of said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

Thus, in a further aspect, the present invention provides the topical composition of the present invention for use in a method of topically treating a disease or disorder mediated by PDE5 activity and/or NO related endothelial dysfunction, preferably of a disease or disorder of the skin mediated by PDE5 activity and/or NO related endothelial dysfunction, in a subject, preferably in a human. Preferably, said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides the topical composition of the present invention for use in a method of topically treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides a method for treating a disease or disorder mediated by PDE5 activity and/or NO related endothelial dysfunction, preferably of a disease or disorder of the skin mediated by PDE5 activity and/or NO related endothelial dysfunction, in a subject, preferably in a human, wherein said method comprises topically administering the topical composition of the present invention to said subject, preferably to said human.

In another aspect, the present invention provides a method for treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle, wherein said method comprises topically administering the topical composition of the present invention to said subject, preferably to said human. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In another aspect, the present invention provides for the use of the topical composition of the present invention for the preparation of a medicament for topically treating a disease or disorder mediated by PDE5 activity and/or NO related endothelial dysfunction, preferably of a disease or disorder of the skin mediated by PDE5 activity and/or NO related endothelial dysfunction, in a subject, preferably in a human, wherein said method comprises topically administering the topical composition of the present invention to said subject, preferably to said human.

In another aspect, the present invention provides for the use of the topical composition of the present invention for the preparation of a medicament for topically treating a disease or disorder in a subject, preferably in a human, wherein said disease or disorder is selected from (a) treating a skin disease, preferably skin aging or steroid-induced skin atrophy; (b) treating erectile dysfunction; (c) preventing or treating hair loss, wherein said hair loss is preferably alopecia, and wherein further preferably said alopecia is androgenetic alopecia (AGA) or chemotherapy-induced alopecia (CIA) or alopecia areata; or (d) promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle, wherein said method comprises topically administering the topical composition of the present invention to said subject, preferably to said human. Very preferred, said disease or disorder is preventing or treating hair loss, wherein said hair loss is alopecia, and wherein preferably said alopecia is androgenetic alopecia (AGA) or chemotherapy-induced alopecia (CIA) or alopecia areata. Very preferred, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

In a further aspect, the present invention relates to a pharmaceutical composition for preventing or treating hair loss or promoting hair growth, comprising a compound of formula I, a derivative thereof or a pharmaceutically acceptable salt thereof as an active ingredient, wherein preferably said compound of formula I is Compound 1 or Compound 2, and wherein further preferably said compound of formula I is Compound 1.

In a preferred embodiment, said disease or disorder is preventing or treating hair loss. In a preferred embodiment, said disease or disorder is preventing or treating hair loss, wherein said hair loss is alopecia. In a preferred embodiment, said disease or disorder is preventing or treating hair loss, wherein said hair loss is alopecia, and wherein said alopecia is androgenetic alopecia (AGA) or chemotherapy-induced alopecia (CIA) or alopecia areata. In a preferred embodiment, said alopecia is androgenetic alopecia (AGA). In a preferred embodiment, said alopecia is chemotherapy-induced alopecia (CIA). In a preferred embodiment, said alopecia is alopecia areata.

In a preferred embodiment, said disease or disorder is promoting hair growth via (i) inducing anagen of hair follicles; (ii) promoting melanogenesis; (iii) increasing the number of hair follicles; (iv) increasing skin thickness; (v) promoting angiogenesis; (vi) proliferating cells in outer root sheath; and/or (vii) enhancing blood flow in the hair follicle.

### EXAMPLES

Synthesis of Compounds of formula I: Compounds of formula I as well as their syntheses are disclosed in WO/2018/215433, the disclosure of which is herewith incorporated by reference in its entirety. In particular Examples 7, 14, 20, 55, 57, 103, 106 and 107 of WO/2018/215433 contain a detailed description of the preparation of preferred compounds of formula I.

Materials: The following materials were used to prepare exemplary compositions described herein, all of said materials are known to the skilled person in the art and are further described in detail in Handbook of Pharmaceutical Excipients published jointly by the American Pharmaceutical Association and The Pharmaceutical Society of Great Britain (eds RC Rowe, PJ Sheskey and ME Quinn; Pharmaceutical Press, London, 2009): Transcutol (diethylene glycol monoethylether [IUPAC: 2-(2-ethoxyethoxy)ethanol], purity ≥99%, Sigma Aldrich); Kolliphor RH40 (Sigma Aldrich, polyoxyl 40 hydrogenated castor oil); PEG-400 (C₂ₙH₄ₙ₊₂O₁₊ᵢ; n=8.2 to 9.1; VWR); Ethanol BioUltra (Sigma Aldrich).

Analytical HPLC: HPLC-MS was used for the analysis of the exemplary compositions described herein. The content of compound 1 in the compositions was within the predefined range of acceptance.

Equipment: AGILENT 1100, MS SCIEC API 4000 Q-TRAP MASS SPECTROMETER

Analytical conditions: Column: Zorbax XBD-C₁₈ Column, 4.6*50 mm, 3.5 μm. Column temp.: 40° C., Mobile phase A: Water/acetonitrile (95/5:v/v)+0.1% formic acid in water, Mobile phase B: Water/acetonitrile (5/95:v/v)+0.1% formic acid in water, Flow 0.4 mL/min, Injection vol.: 20 μL (single injection), detection: 250 nm wavelength.

| Gradient Table - Time [min] | % A | % B |
|---|---|---|
| 1.50 | 70.0 | 30.0 |
| 1.00 | 5.0 | 95.0 |
| 5.00 | 5.0 | 95.0 |
| 5.50 | 95.0 | 5.0 |
| 6.2 | 95.0 | 5.0 |
| 6.50 | 70.0 | 30.0 |
| 7.50 | 70.0 | 30.0 |

All exemplary compositions were clear solutions by visual inspection.

### Example 1

### Preparation of Composition Vehicles

### A. Preparation of Vehicle Formulation A

For 100 ml of Vehicle Composition A 70 ml PEG-400 was mixed with 30 ml ethanol and the solution was stirred 10 min.

### B. Preparation of Vehicle Composition B

For 100 ml of Vehicle Composition B 10 ml PEG-400 was mixed with 10 ml Transcutol and 30 ml ethanol and the solution was stirred 10 min. To the solution 0.130 ml Kolliphor was added and stirred for 24 h at room temperature. After 24 h 50 ml water was added and stirred for 15 min.

### Example 2

### Preparation of Inventive Compositions

### A. Preparation of Topical Composition A1

For 100 ml of Topical Composition A1, 70 ml PEG-400 was mixed with 30 ml ethanol and the solution was stirred 10 min. To the solution, 0.2 mg (0.3 μmol) of Compound 1 was added and stirred for 24 h at RT.

### B. Preparation of Topical Composition A2

For 100 ml of Topical Composition A2, 70 ml PEG-400 was mixed with 30 ml ethanol and the solution was stirred 10 min. To the solution, 2 mg (3 μmol) of Compound 1 was added and stirred for 24 h at RT.

### C. Preparation of Topical Composition A3

For 100 ml of Topical Composition A3, 70 ml PEG-400 was mixed with 30 ml ethanol and the solution was stirred 10 min. To the solution, 20 mg (30 μmol) of Compound 1 was added and stirred for 24 h at RT.

### D. Preparation of Topical Composition A4

For 100 ml of Topical Composition A4, 70 ml PEG-400 was mixed with 30 ml ethanol and the solution was stirred 10 min. To the solution, 0.4 mg (0.6 μmol) of Compound 1 was added and stirred for 24 h at RT.

### E. Preparation of Topical Composition A5

For 100 ml of Topical Composition A5, 70 ml PEG-400 was mixed with 30 ml ethanol and the solution was stirred 10 min. To the solution, 2 mg (3.5 μmol) of Compound 2 was added and stirred for 24 h at RT.

### F. Preparation of Topical Composition A6

For 100 ml of Topical Composition A3, 70 ml PEG-400 was mixed with 30 ml ethanol and the solution was stirred 10 min. To the solution, 20 mg (35 μmol) of Compound 2 was added and stirred for 24 h at RT.

### G. Preparation of Topical Composition B1

For 100 ml of Vehicle Composition B1, 10 ml PEG-400 was mixed with 10 ml Transcutol and 30 ml ethanol and the solution was stirred 10 min. To the solution, 0.2 mg (0.3 μmol) of Compound 1 and 0.130 ml Kolliphor was added and stirred for 24 h at RT. After 24 h 50 ml water was added and stirred for 15 min.

### Example 3

### Topical, on Skin Administration of Compound 1 and Effects on Hair Growth in C57BL/6 Mouse Model of Alopecia In Vivo

Dorsal hair of C57BL/6 mouse is known to have a time synchronized hair cycle. From about 18-21 days of age (2-3 weeks old) and 47-95 days of age (6-14 weeks old), the dorsal hair are in the telogen/quiescent phase (visually pink skin) [Lee B H, Lee J S, Kim Y C. Hair growth-promoting effects of lavender oil in C57BL/6 mice. Toxicol Res. (2016) 32(2):103-108; Choi H I, Kang B M, Jang J, Hwang S T, Kwon O. Novel effect of sildenafil on hair growth. Biochem Biophys Res Commun. (2018) 505(3):685-691]. C57BL/6 mouse therefore is a well-established model for studying hair growth promotion.

### Animals

Male and female C57BL/6 mice of 6 to 9 weeks of age in stable telogen phase of hair growth cycle were used (obtained from Vivo Bio Tech Ltd, Telangana, India). The details of different treatment groups are provided in Table 1.

TABLE 1
Details of different treatment groups

| | | | | No. of |
|---|---|---|---|---|
| | | Concentration of | No. of animals | animals |
| Treatment group no | | compounds | male | female |
| 1 | Positive | Minoxidil 5% (w/v) | 5 | — |
| | Control | Minoxidil 2% (w/v) | — | 5 |
| 2 | Compound 1 | 0.0002% (w/v) | 5 | 5 |
| | Composition A1 | | | |
| 3 | Compound 1 | 0.002% (w/v) | 5 | 5 |
| | Composition A2 | | | |
| 4 | Compound 1 | 0.02% (w/v) | — | 5 |
| | Composition A3 | | | |
| 5 | Vehicle | PEG-400: EtOH/7:3 | 5 | 5 |
| | Composition A | | | |

### Compound Treatment Application

50 μL of a clear solution of respective 0.02%, 0.002% and 0.0002% (w/v) concentrations of Cpd 1 in the corresponding vehicle (PEG-400:EtOH/7:3) (PEG-400: Central Drug House Ltd. Dehli, India; Ethanol: Changshu Hongsheng fine chemicals Co. Ltd. China) and the positive control, 2% solution (female) or 5% (male) (w/v) Minoxidil (Chemieliva, China) in the corresponding vehicle (PEG-400:EtOH/7:3) were topically applied to the clipped area on the dorsal back of the mice. The solutions covered a circular area of approximately 2 cm². The treatment regimen was twice daily application for 5 days/week for total of 4 weeks.

### Induction of Anagen Hair Growth in C57BL/6 Mice

The selected animals on day 1 weighted on average 20.05 g±0.17 ranging from 19.8 to 20.2 g. The hair on the dorsal back (approx. 4×4 cm²) of these animals were clipped without touching the skin of the animal, using an electric clipper, on day 0 before the treatment with Cpd 1 and the hair was collected. The treatment followed by visual analysis for anagen onset, skin color change and appearance of any hair growth were monitored. After completion of the experiment, the dorsal skin of all the animals was removed and visual melanogenesis was reported. The skin sections were then preserved in 10% formalin solution for further histopathology analysis.

Animals were observed during 30 days and the hair growth score was recorded on daily basis. The criteria for scoring of hair growth is as follows: no hair growth, pink skin—score 0; skin colour changes from pink to grey/light grey without visible hair growth—score 0.5 onset of anagen (skin colour changes from grey/light grey to dark grey/black without visible hair growth)—score 1; sparse hair growth—score 1.5; diffuse short hair growth—score 2, moderate hair growth—score 2.5 and dense, normal coat hair—score 3 (Lee B H, Lee J S, Kim Y C. Hair growth-promoting effects of lavender oil in C57BL/6 mice. Toxicol Res. (2016) 32(2):103-108).

Photographs of the animals were captured before the initiation and during the course of the study. To maintain the integrity of the study, animals which displayed sudden abrupt hair growth on random areas of dorsal clipped skin, were considered as outlier and were not included in the experiment.

### Histology and Immunohistochemistry

After completion of the experiments all animals were sacrificed by euthanasia (Choi H I, Kang B M, Jang J, Hwang S T, Kwon O. Novel effect of sildenafil on hair growth. Biochem Biophys Res Commun. (2018) 505(3):685-691). The dorsal skin of each animal was then removed by peeling. The peeled skin was spread and checked visually for the induction of melanogenesis indicated by visual blackening on the reverse side of the skin. The skin samples were evenly spread on a pre-labelled glass slide and kept in in 10% formalin until further use. Later, the skin samples were embedded in paraffin and sectioned at 5 microns using a microtome and the corresponding slides per animal were prepared. The slides obtained were used for Haematoxylin & Eosin (H&E) staining and in the transverse and longitudinal sections' photomicrographs data, the total number of follicles in the dermis and the sub cutis were determined for each animal. The skin thickness was measured using Image J 1.44 software.

### Statistical Analysis

The statistical analyses were performed using GraphPad Prism 9.0.0 software. Statistical significance was determined by One-way or Two-Way ANOVA followed by Tukey's multiple comparison test. The results are presented as means+/−SEM from the number of animals per group. The statistical significance was considered at P<0.05.

### Results

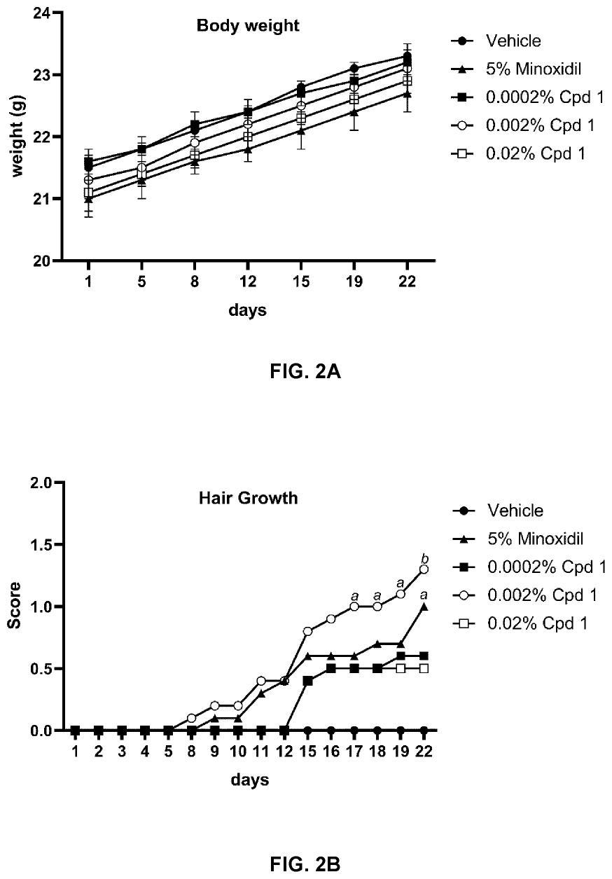

Male mice: During the study, all animals were observed for skin colour change from pink (telogen) to grey/black (anagen) and appearance of new hair re-growth. No adverse effects were observed in relation to general health or body weight of treated male animals with Cpd 1 compared to vehicle group (FIG. 2A). From day 5 on, hair growth was significantly higher in 0.002% Cpd 1-treated group compared to vehicle (FIG. 2B). Moreover, 0.002% Cpd 1-treated group showed significant increase in hair growth from day 15 compared to 5% Minoxidil. Throughout the study no anagen phase was observed in the vehicle group (FIG. 2B).

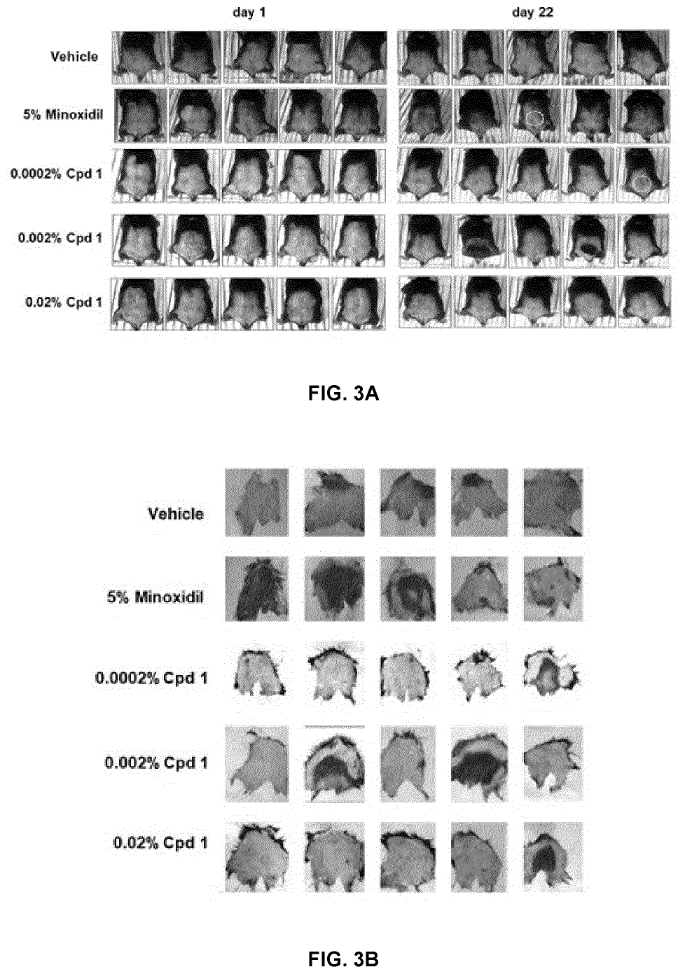

It was found that 0.002% Cpd 1-treated mice exhibited skin colour change from pink to light grey at day 10 after hair growth induction, indicating a transition from the telogen to the anagen phase. Also, in 0.002% Cpd 1-treated group hair growth was significantly higher than the vehicle group at day 22 (FIG. 3A). At the end of the study the skin biopsies were collected and observed for signs of visual melanogenesis at the site of compound application. Treatment with 0.002% Cpd 1 led to appearance of melanogenesis in peeled skin of 2/5 animals. No visual melanogenesis was observed in the vehicle (FIG. 3B).

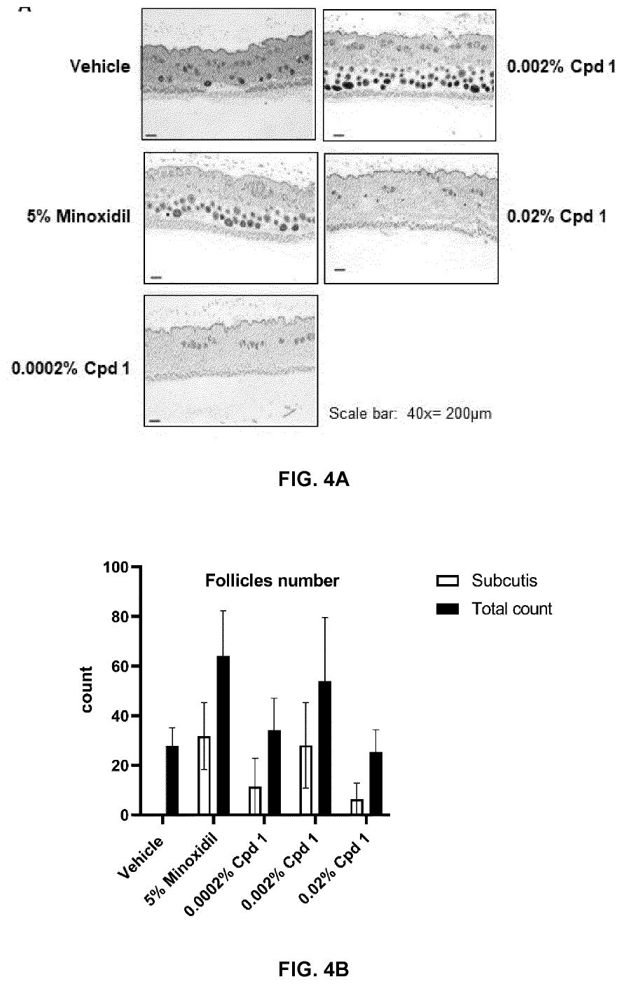

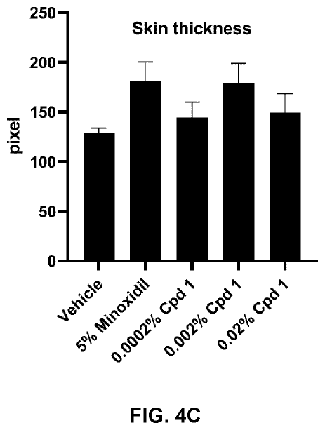

Mouse skin thickness changes based on the hair cycle, being maximal in the anagen phase and becoming thinner when reaching the telogen phase. Histological studies showed more anagen hair follicles in Cpd 1-treated groups (FIG. 4A). Cpd 1 led to the appearance of considerable higher number of follicles in the subcutis layer of the skin and total follicle counts when compared to the control group (FIG. 4B). Furthermore, skin thickness increased after 0.002% Cpd 1 treatment when compared to vehicle group (FIG. 4C).

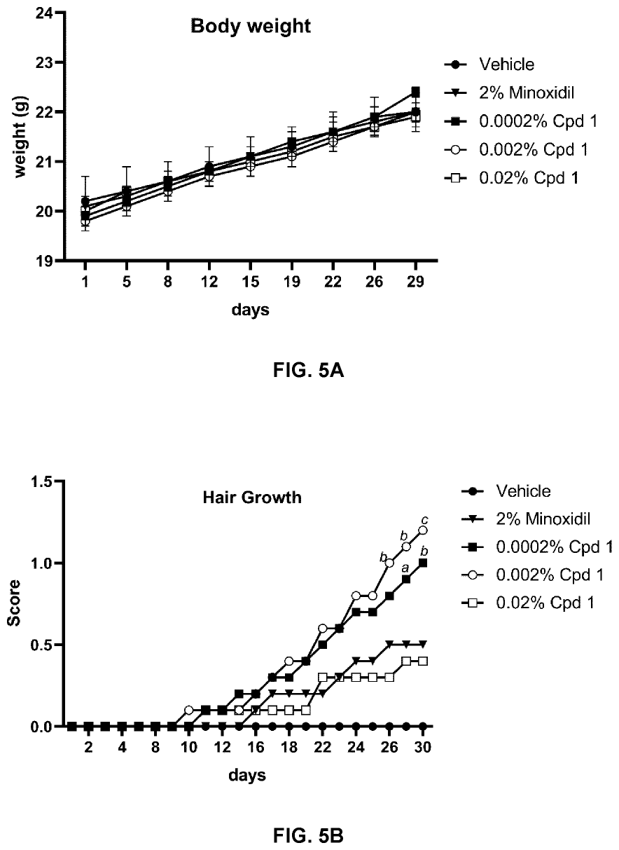

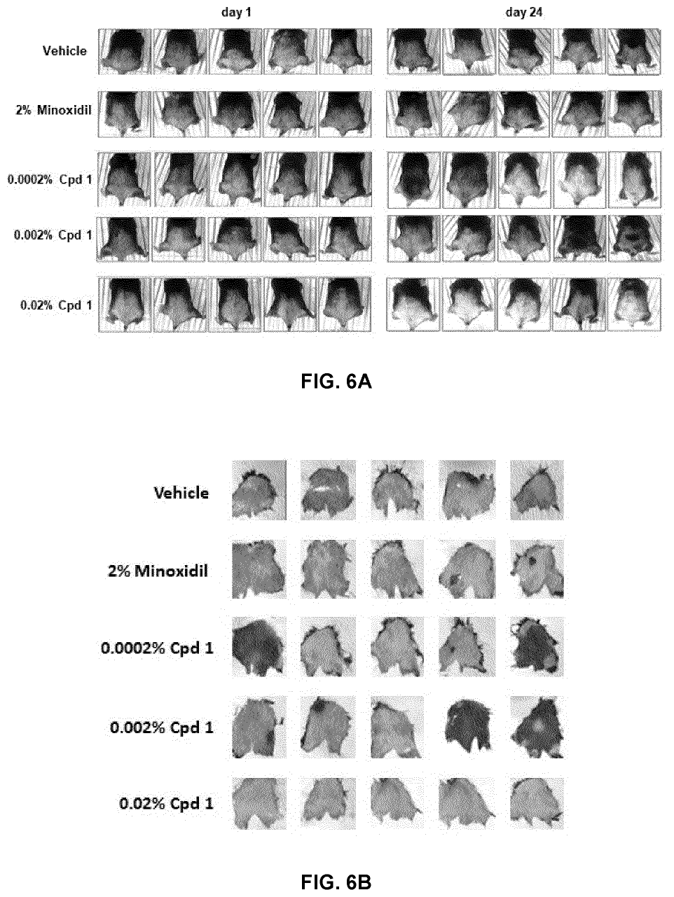

Female mice: During the study, all animals were observed for skin colour change from pink (telogen) to grey/black (anagen) and appearance of new hair re-growth. No adverse effects were observed in relation to general health or body weight of treated animals compared to vehicle group (FIG. 5A) From day 26 on, hair growth was significantly higher in the 0.002% Cpd 1-treated group compared to the vehicle group (FIG. 5B). Moreover, at day 30 the maximal hair growth score was observed in mice treated with 0.002% Cpd 1, followed by low dose treatment groups and Minoxidil group (FIG. 5B). Throughout the 30-day study no anagen phase was observed in the vehicle group (FIG. 5B). It was found that 0.002% Cpd 1-treated mice exhibited skin colour change from pink to light grey at day 10 after hair growth induction, indicating a transition from the telogen to the anagen phase. At day 24, the area of black skin was larger in Cpd 1 treated mice compared to the control groups (FIG. 6A). At the end of the study the skin biopsies were collected and observed for signs of visual melanogenesis at the site of compound application. Treatment with 0.0002% and 0.002% Cpd 1 led to appearance of melanogenesis in peeled skin of 2/5 animal, respectively. No visual melanogenesis was observed in both vehicle and positive control groups (FIG. 6B).

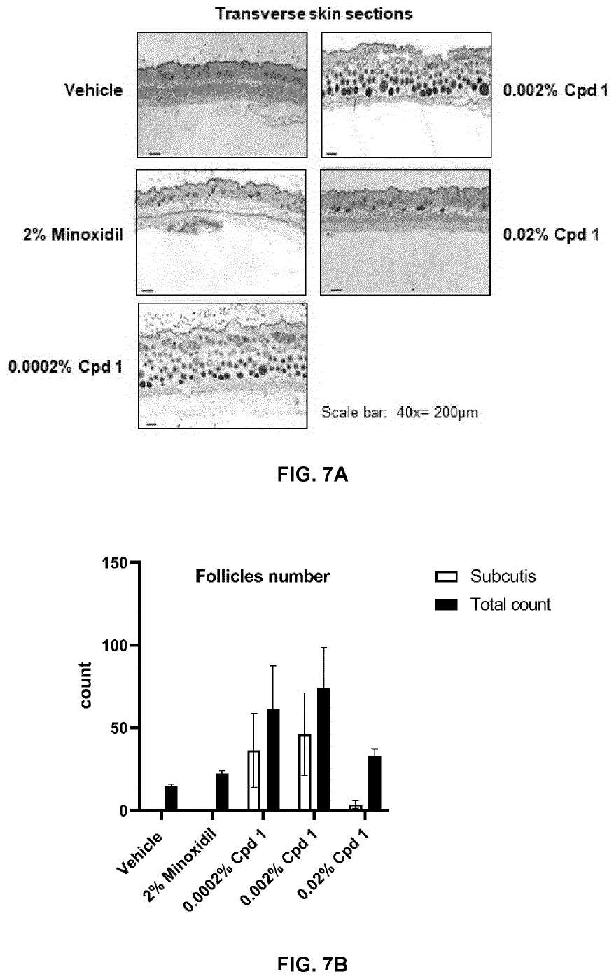

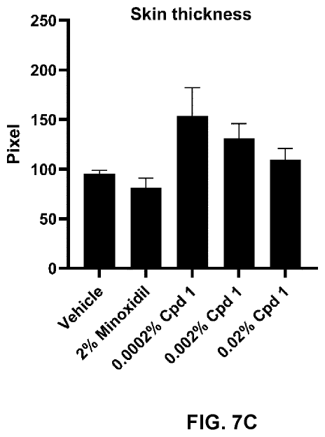

Histological studies showed more anagen hair follicles in Cpd 1-treated groups 0.0002% and 0.002% compared to vehicle and 2% Minoxidil (FIG. 7A). Cpd 1 led to the appearance of considerable higher number of follicles in the subcutis layer of the skin and total follicle counts when compared to the control group (FIG. 7B). Furthermore, skin thickness increased after Cpd 1 treatment when compared to vehicle and 2% Minoxidil groups (FIG. 7C).

### Example 4

### Topical, on Skin Administration of Inventive Cpd 2 and the Effects on Hair Growth in C57BL/6 Mouse Model of Alopecia In Vivo

The experimental set up and analysis were performed as described in Example 1

TABLE 2
Details of different treatment groups

| | | | | No. of |
|---|---|---|---|---|
| | | Concentration of | No. of animals | animals |
| Treatment Group no | | compounds | male | female |
| 1 | Positive | Minoxidil 5% (w/v) | 5 | — |
| | Control | Minoxidil 2% (w/v) | — | 5 |
| 2 | Cpd 2 | 0.002% (w/v) | — | 5 |
| | Composition A5 | | | |
| 3 | Cpd 2 | 0.02% (w/v) | — | 5 |
| | Composition A6 | | | |
| 4 | Vehicle | (PEG400:EtOH/7:3) | 5 | 5 |
| | Composition A | | | |

### Compound Treatment Application

50 μL of a clear solution of respective 0.02%, 0.002% (w/v) concentrations of Cpd 2 in the corresponding vehicle (PEG-400:EtOH/7:3) (PEG-400: Central Drug House Ltd. Dehli, India; Ethanol: Changshu Hongsheng fine chemicals Co. Ltd. China) and the positive control, 2% solution (female) or 5% (male) (w/v) Minoxidil (Chemieliva, China) in the corresponding vehicle (PEG-400:EtOH/7:3) were topically applied to the clipped area on the dorsal back of the mice. The solutions covered a circular area of approximately 2 cm². The treatment regimen was twice daily application for 5 days/week for total of 4 weeks.

### Results

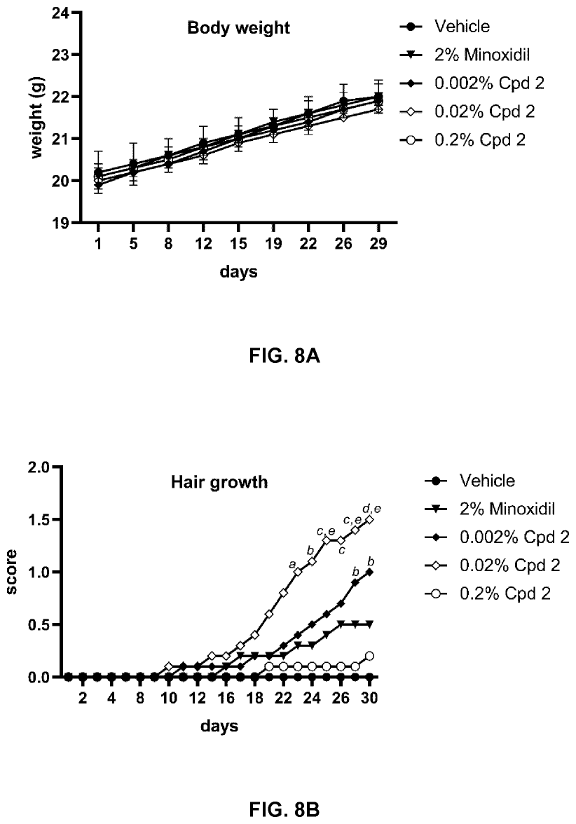

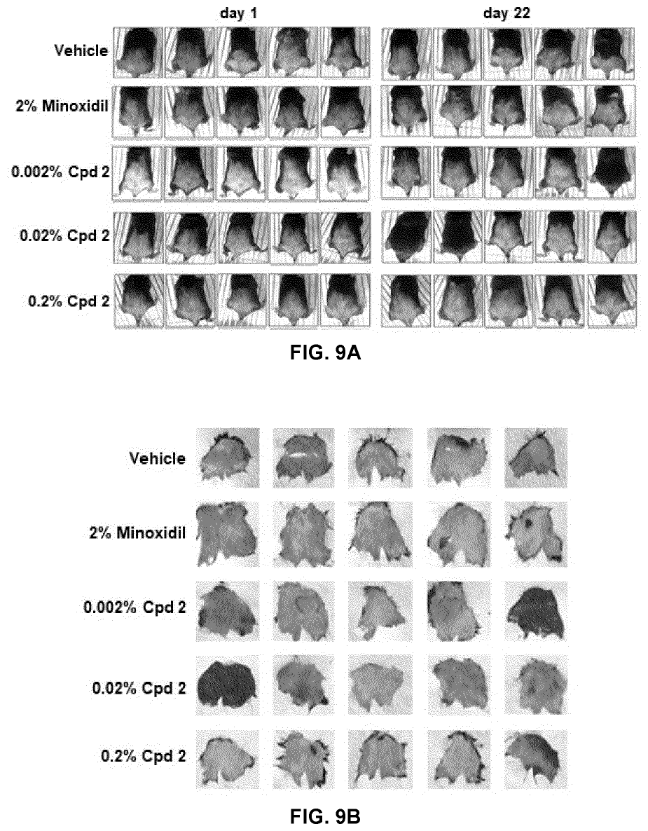

Female mice: During the study, all animals were observed for skin colour change from pink (telogen) to grey/black (anagen) and appearance of new hair re-growth. No adverse effects were observed in relation to general health or body weight of treated animals compared to vehicle group (FIG. 8A). From day 23 on, hair growth was significantly higher in treated group 2 compared to vehicle. Moreover, treated group 2 showed significant increase in hair growth at days 25, 29 and 30 compared to 2% Minoxidil. In addition, at day 30 the maximal hair growth score was observed in mice in treated group 2 followed by low dose treatment groups and Minoxidil group (FIG. 8B). Throughout the 30-day study no anagen phase was observed in the vehicle group (FIG. 8B). It was found that treated group 2 mice exhibited skin colour change from pink to light grey at day 10 after hair growth induction, indicating a transition from the telogen to the anagen phase. Also, in treated group 3 hair growth was significantly higher than the vehicle group at day 29 and 30. At day 30, the area of black skin was larger in Cpd 2 treated mice compared to the control groups (FIG. 9A). At the end of the study the skin biopsies were collected and observed for signs of visual melanogenesis at the site of compound application. Treatment with 0.002% and 0.02% Cpd 2 led to appearance of melanogenesis in peeled skin of 1/5 animal, respectively. No visual melanogenesis was observed in both vehicle and positive control groups (FIG. 9B).

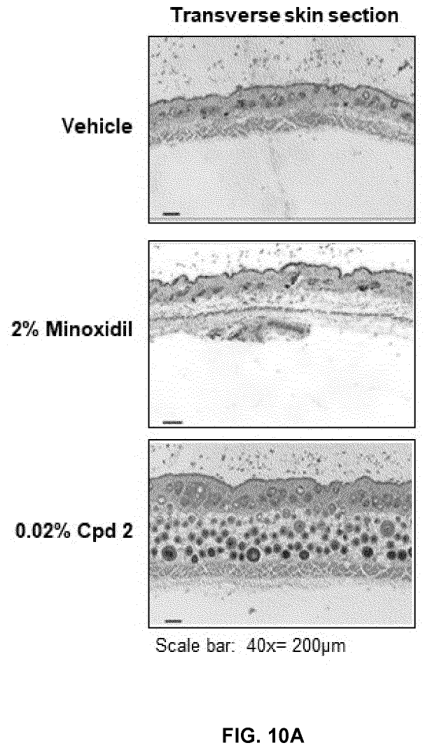

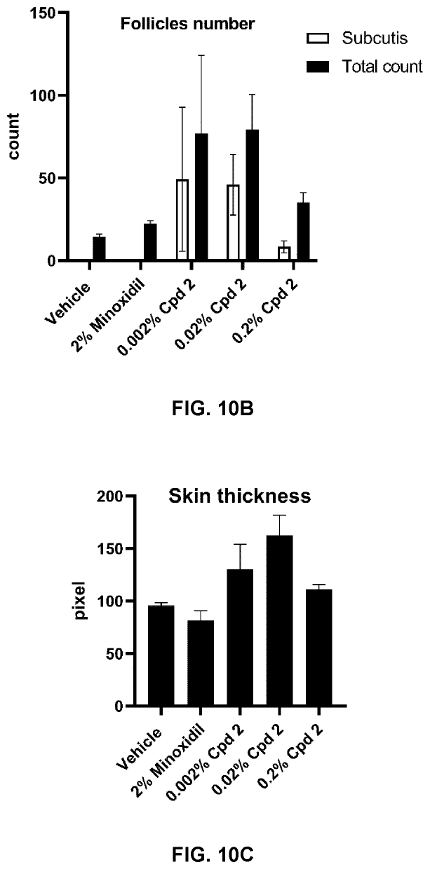

Mouse skin thickness changes based on the hair cycle, being maximal in the anagen phase and becoming thinner when reaching the telogen phase. By comparing skin thickness of experimental versus control groups, it was possible to evaluate anagen induction. Histological studies showed more anagen hair follicles in Cpd 2-treated groups compared to the control groups (FIG. 10A). Cpd 2 treatment led to the appearance of considerable higher number of follicles in the subcutis layer of the skin and total follicle counts when compared to the control group (FIG. 10B). Furthermore, skin thickness increased after 0.02% Cpd 2 treatment when compared to vehicle and 2% Minoxidil groups (FIG. 10C).

### Example 5

### Topical, on Skin Administration of Inventive Compound and its Effects on a Preventive Model of Chemotherapy-Induced Alopecia (CIA) In Vivo

### Animals

Female C57BL/6 mice (Vivo Bio Tech Ltd, Telangana, India) of 6 to 9 weeks of age in stable telogen phase of hair growth cycle were used. The details of different treatment groups are provided in Table 3.

TABLE 3
Details of the different treatment groups.

| Treatment group no | | Treatment group | No. of animals |
|---|---|---|---|
| 1 | Positive Control | Minoxidil 2% (w/v) | 7 |
| 2 | Cpd 1 | 0.0004% (w/v) | 7 |
| | Composition A4 | | |
| 3 | Cpd 1 | 0.002% (w/v) | 7 |
| | Composition A2 | | |
| 4 | Vehicle | (PEG400:EtOH/7:3) | 7 |
| | Composition A | | |
| 5 | Disease Control | Cyclophosphamide induced CIA | 6 |

### Compound Treatment Application

100 μL of a clear solution of respective 0.0004%, 0.002% (w/v) concentrations of Cpd 1 in the corresponding vehicle (PEG-400:EtOH/7:3) (PEG-400: Central Drug House Ltd. Dehli, India; Ethanol: Changshu Hongsheng fine chemicals Co. Ltd. China) and the positive control, 2% solution (w/v) Minoxidil (Chemieliva, China) in the corresponding vehicle (PEG-400:EtOH/7:3) were topically applied to the depilated area on the dorsal back of female mice. The solutions covered a circular area of approximately 4 cm². The treatment regimen was twice daily for 7 days before Cyclophosphamide injection and afterwards for 3 weeks.

### C57BL/6 Mouse Model for Chemotherapy-Induced Alopecia (CIA)

Animals (female) on day 1 weighted on average 18.8 g+0.2 ranging from 18.5 to 19.3 g. The animals were exposed by gentle clipping of hair from the dorsal back using an electric clipper. The following day, animals with clear skin were depilated using a depilatory cream. To evaluate the preventive potential of Cpd 1 in the chemotherapy-induced alopecia mouse model, animals were treated topically with 0.0004% and 0.002% Cpd 1, 2% Minoxidil and vehicle from day 1 onwards on the dorsal depilated skin with twice daily application regimen until day 10, when all the animals have attained the anagen stage. On day 11 after depilation, no application of the Cpd 1 or vehicle was done. This was considered a wash-out period. On day 12 after depilation, the animals were given intraperitoneal injection of cyclophosphamide (CYP-HiMedia Laboratories, Mumbai, India) at the dose of 150 mg/kg body weight. One day of wash-out period was given to the animals before restarting the treatment with Cpd 1. The treatment regimen was reinitiated with 0.0004% and 0.002% Cpd 1, 2% Minoxidil and vehicle in the respective groups for further 3 weeks. All the animals were observed from day 1 (experiment start) till day 30 (experiment termination and animal sacrifice day) and the hair growth score was recorded on a daily basis. The criteria for scoring of hair growth is as follows: no hair growth, pink skin—score 0; skin colour changes from pink to grey/light grey without visible hair growth—score 0.5; onset of anagen (skin colour changes from grey/light grey to dark grey/black without visible hair growth)—score 1; sparse hair growth—score 1.5; diffuse short hair growth—score 2, moderate hair growth—score 2.5 and dense, normal coat hair—score 3 (Lee B H, Lee J S, Kim Y C. Hair growth-promoting effects of lavender oil in C57BL/6 mice. Toxicol Res. (2016) 32(2):103-108). Photographs of the animals were captured before the initiation and during the course of the study. Photographs of the animals were taken before initiation and during the course of the study.

### Histology and Immunohistochemistry

After completion of the experiments all animals were sacrificed by euthanasia (Choi H I, Kang B M, Jang J, Hwang S T, Kwon O. Novel effect of sildenafil on hair growth. Biochem Biophys Res Commun. (2018) 505(3):685-691). The dorsal skin of each animal was then removed by peeling. The peeled skin was spread and checked visually for the induction of melanogenesis indicated by visual blackening on the reverse side of the skin. The skin samples were evenly spread on a pre-labelled glass slide and kept in in 10% formalin until further use. Later, the skin samples were embedded in paraffin and sectioned at 5 microns using a microtome and the corresponding slides per animal were prepared. The slides obtained were used for Haematoxylin & Eosin (H&E) staining and in the transverse and longitudinal sections' photomicrographs data, the total number of follicles in the dermis and the sub cutis were determined for each animal. The skin thickness was measured using Image J 1.44 software. Immunohistochemistry analysis was performed using the CD-31 antibody (Anti-CD31, Endothelial Cell, JC/70A, XBioGenex) and Ki-67 antibody (Rabbit anti-Ki-67, ZytoMED Systems) to observe perifollicular vessel formation and proliferative index in the hair follicles, respectively. A semi-quantitative grading was applied. For CD-31 the scoring was based on the numbers of capillary vessels near the follicle base. Score 1 was assigned to skin sections where one or two capillary vessels near the base of the follicle were observed, whereas more than 2 capillary vessels near follicle base was given a score of 2. For the Ki-67 marker, the presence of positive Ki-67 cells in the outer root sheath cells of a hair follicle scored 1 or 2 depending on the intensity of the staining.

### Results

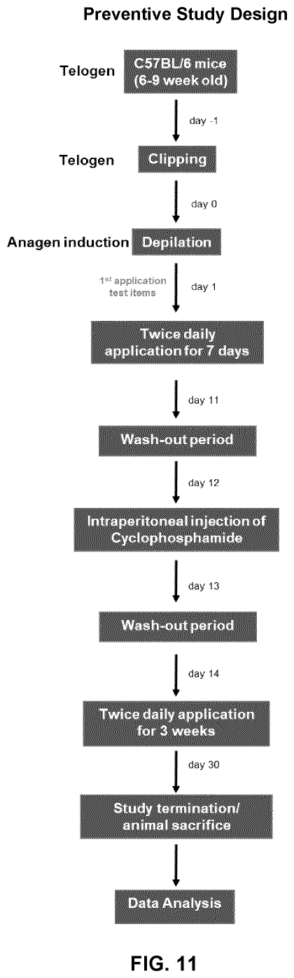

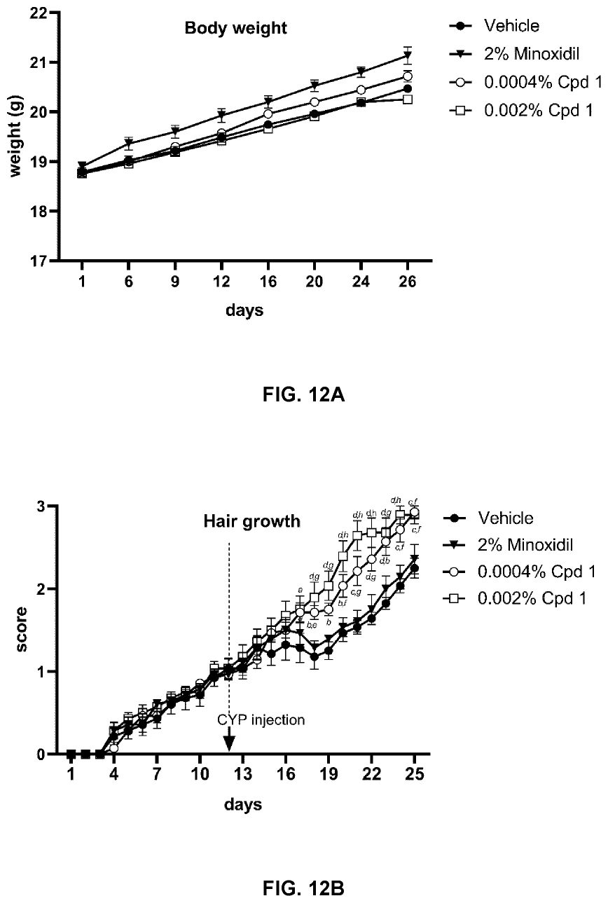

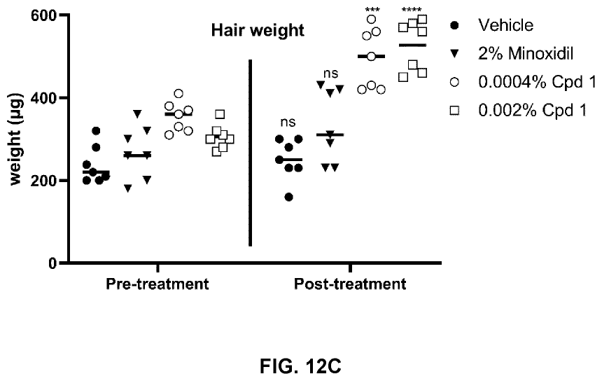

To study the preventive hair growth-promoting potential of Cpd 1 in a cyclophosphamide-induced hair loss mouse model, C57BL/6 female mice in stable telogen phase were used. In this 30-day study, a wash-out period was done before and after CYP administration (FIG. 11A). In each group the animals were observed for the onset of hair loss after administration of CYP. During the course of the study, no signs of behavioral changes, reaction to treatment or adverse effects in relation to general health were observed. Also, no adverse effects on body weight were observed in any of the groups (FIG. 12A).

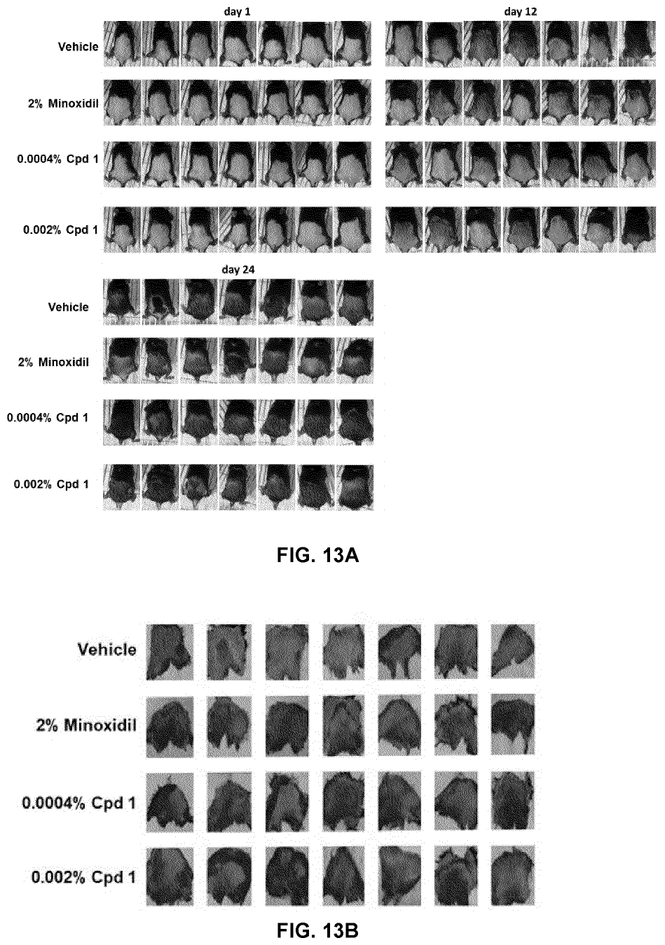

The skin colour of the C57BL/6 mice is pink during the telogen phase and darkens when anagen begins. Before CYP administration the topical application of Compound 1 (0.0004% and 0.002%) led to faster skin color change (transition from telogen to anagen phase) in the majority of the animals when compared to the Minoxidil-treated group by the end of the first week of application. Similar observations were recorded in animals treated with 2% Minoxidil when compared to the vehicle control group. On day 12 (day of CYP injection), all groups had a mean hair growth score of 1. After CYP administration on day 12, there were minor signs of alopecia in animals treated with 0.0004% Cpd 1 and no signs of alopecia in animals treated with 0.002% Cpd 1. In general, no decrease in the mean hair growth was observed in Compound 1 treated groups (FIG. 12B). Hair samples of all the animals were collected before the initiation of the treatment. On the day of sacrifice of the animals, samples of treated animals with hair growth score of 2-3 were collected for hair weight analysis. Approximately 100 hair strands from pre-treatment and post-treatment hair samples were selected from animals in which hair regrowth was observed and hair were weighed. Treatment with 0.0004% and 0.002% TOP-M119 led to significantly increase in hair weight in 7/7 animals compared to the respective pretreatment hair of the same animals (FIG. 12C). It was also observed that the topical application of Cpd 1 led to faster increase in hair growth from day 6/7 onwards compared to Minoxidil and vehicle group. Application of Cpd 1 led to complete hair regrowth in all 7/7 animals (hair growth score of 3). Furthermore, a slightly faster increase in hair growth was observed in animals treated with 0.002% when compared to 0.0004% of Compound 1 (FIG. 13A). Treatment with 2% Minoxidil led to faster attainment of hair growth compared to vehicle. However, complete hair growth was only observed in 3/7 animals (hair growth score of 3) and 4/7 animals showed moderate hair growth (hair growth score of 2-2.5). In the vehicle treated group, 2/7 animals attained full hair growth and 4/7 animals showed spare to moderate hair growth (hair growth score of 2-2.5) and 1 animal did not show hair growth (FIG. 13A).

Additionally, at the end of the study skin biopsies were collected and analysed for sign of visual melanogenesis at the site of the compound application. Treatment with 0.0004% and 0.002% Cpd 1 led to appearance of melanogenesis in peeled skin of 6/7 and 7/7 mice, respectively. Visual melanogenesis was also observed in the peeled skin of 7/7 mice treated with 2% Minoxidil and in 3/7 animals of vehicle group (FIG. 13B).

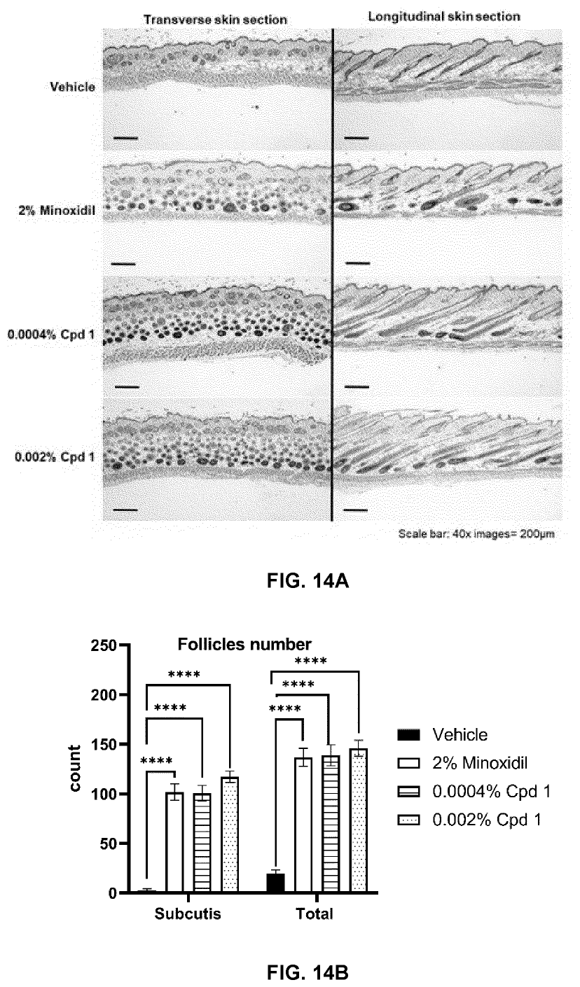

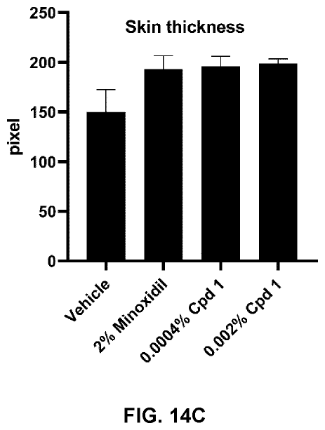

H&E staining showed that the number of hair follicles significantly increased for the Compound 1 treated groups in the subcutis layer of the skin and total follicle counts when compared to vehicle group (FIGS. 14A and B). No significant differences were observed when comparing Cpd 1 with 2% Minoxidil-treated groups.

The longitudinal sections of skin (FIG. 14A panel on the right) were analysed for extent of depth of hair follicles in the animals treated with test compound-induced anagen phase or hair regrowth. The depth and development of dermal papilla and the long hair shaft of hair follicles were observed in the majority of the animals treated with 0.0004% and 0.002% Cpd 1 and 2% Minoxidil. The thickness of the skin showed a slightly increase after treatment with 0.0004% and 0.002% Cpd 1 and 2% Minoxidil compared to vehicle group (FIG. 14C).

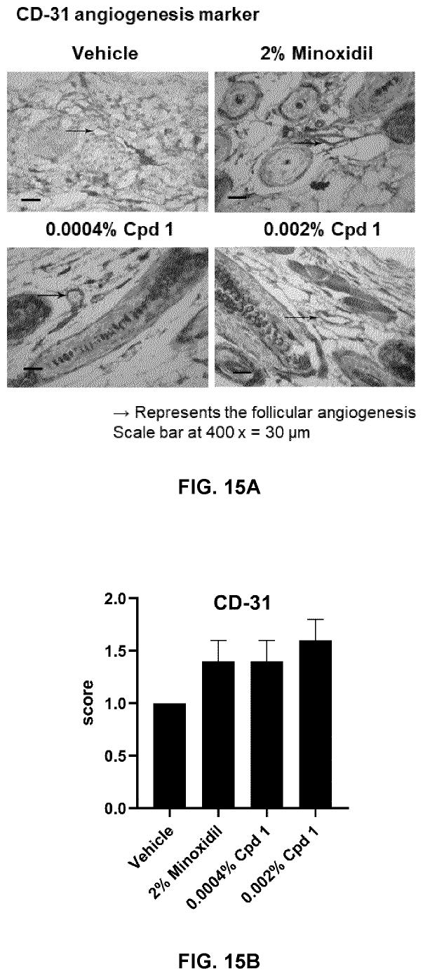

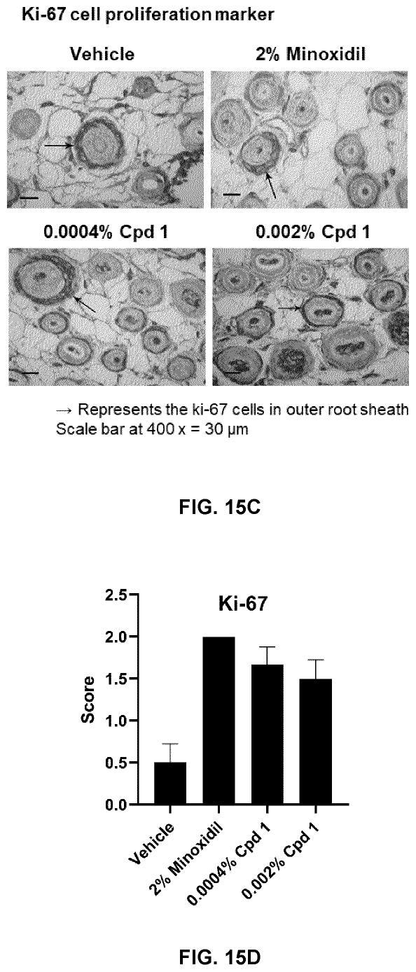

To investigate whether Cpd 1 promotes perifollicular vessel formation and has an effect on cell proliferation immunohistochemistry analysis using CD-31 and Ki-67 antibodies was performed and a semi quantitative grading was reported. Increased number of blood vessels near the periphery of hair follicles were observed after treatment with 0.0004% and 0.002% Cpd 1 with mean score of 1.4 and 1.6, respectively indicating perifollicular angiogenesis. Also, animals treated with 2% Minoxidil showed angiogenesis near follicular region with a mean score of 1.4; while vehicle group had a mean score of 0.8 (FIGS. 15A and B).

The presence of Ki-67 positive cells near the root sheath of hair follicles was higher in both Cpd 1 treatments (0.0004% and 0.002%) compared to vehicle group with a mean score of 1.4 and 1.7, respectively. Treatment with 2% Minoxidil also led to Ki-67 positive cells near the follicular region with a score of 1.1. Vehicle control animals were found to have a weak ring of Ki-67 positive cells near the root sheath of hair follicle with a score of 0.4 (FIGS. 15C and D).

### Example 6

### In Vitro Safety Pharmacology of Compound 1

In vitro pharmacological profiling is increasingly being used earlier in the drug discovery process to identify undesirable off-target activity profiles that could hinder or halt the development of candidate drugs or even lead to market withdrawal if discovered after a drug is approved (Bowes N, et. al., Nature Reviews Drug Discovery 11(12):909-22). Thus, the Off-target selectivity of Compound 1 at 100 nM was tested in (radio)ligand binding and enzymatic assays against a battery of 44 targets (SafetyScreen44™, atEurofins Cerep, France). Compound 1 (at 100 nM) showed no significant responses at all 44 targets, as defined by <50% inhibition or stimulation to any of the targets.

TABLE 4
List of 44 targets tested in (radio)ligand binding and enzymatic
assays at Eurofins Cerep, France (SafetyScreen44 ™)

| Target | Species | Target | Species |
|---|---|---|---|
| Adenosine A2A | human | μ (MOP) | human |
| Adrenergic a1A | human | 5-HT1A | human |
| Adrenergic a2A | human | 5-HT1B | human |
| Adrenergic b1 | human | 5-HT2A | human |
| Adrenergic b2 | human | 5-HT2B | human |
| BZD (central) | | 5-HT3 | human |
| CB1 | human | GR Glucocorticoid Receptor | human |
| CB2 | human | AR Androgen Receptor | human |
| CCK1 (CCKA) | human | Vasopressin Receptor V1 a | human |
| Dopamine D1 | human | Ca²⁺ channel (L-dihydropyridine | human |
| | | site) | |
| Dopamine D2S | human | Potassium channel hERG | human |
| Endothelin ETA | human | Kv channel | human |
| NMDA | human | Na⁺ channel (site 2) | human |
| Histamine H1 | human | Norepinephrine transporter | human |
| Histamine H₂ | human | Dopamine transporter | human |
| MAO-A | human | 5-HT transporter | human |
| Muscarinic M1 | human | COX1 | human |
| Muscarinic M2 | human | COX2 | human |
| Muscarinic M3 | human | PDE3A | human |
| N neuronal α4ß2 | human | PDE4D2 | human |
| δ (DOP) | human | Lck kinase | human |
| κ (KOP) | human | Acetylcholinesterase | human |

1. A topical composition comprising a compound of formula I or a pharmaceutically acceptable salt thereof 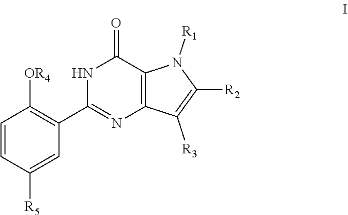
wherein
R₁ is C₁-C₃alkyl, preferably methyl or ethyl, further preferably ethyl;
R₂ is H, CHO or CH═N—OH, preferably H, CHO or (E)-CH═N—OH, further preferably H;
R₃ is C₁-C₄alkyl, preferably ethyl or propyl, further preferably n-propyl;
R₄ is C₁-C₆alkyl, preferably ethyl or propyl, further preferably n-propyl;
R₅ is SO₂NR₁₃R₁₄, wherein R₁₃ and R₁₄ are together with the nitrogen atom to which they are attached form a heterocyclic ring, wherein said heterocyclic ring is selected from piperidine and piperazine, wherein said heterocyclic ring is substituted with at least one R₁₅, and wherein said R₁₅ is independently selected from C₁-C₆alkyl optionally substituted with OH, ONO₂, wherein at least one of said R₁₅ comprises at least one ONO₂ moiety;
wherein said topical composition comprises said compound of formula I, preferably said Compound 1, from about 0.0001% (w/v) to about 0.05% (w/v).

2. The topical composition of claim 1, wherein R₁ is methyl or ethyl; R₂ is H; R₃ is ethyl or n-propyl; and R₄ is ethyl or n-propyl.

3. The topical composition of claim 1 or claim 2, wherein R₁ is ethyl; R₂ is H; R₃ is n-propyl; and R₄ is n-propyl.

4. The topical composition of any one of the claims 1 to 3, wherein said compound of formula I is a compound selected from

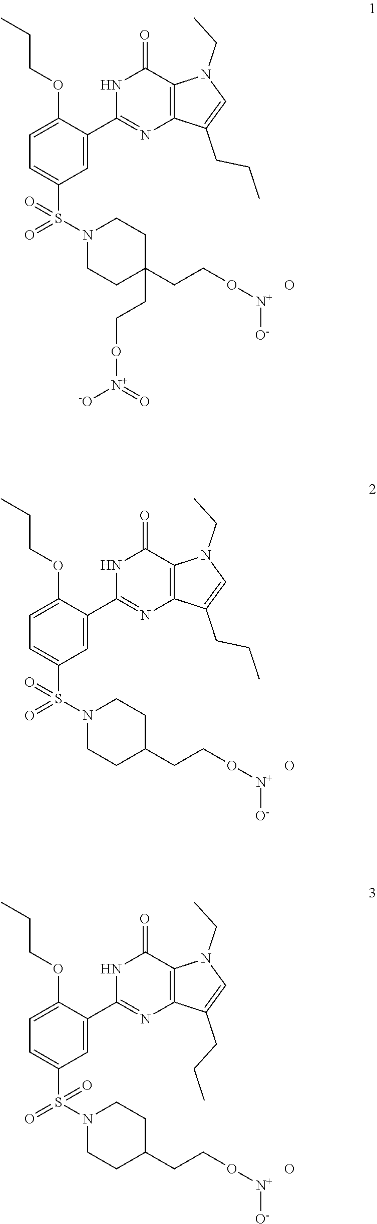

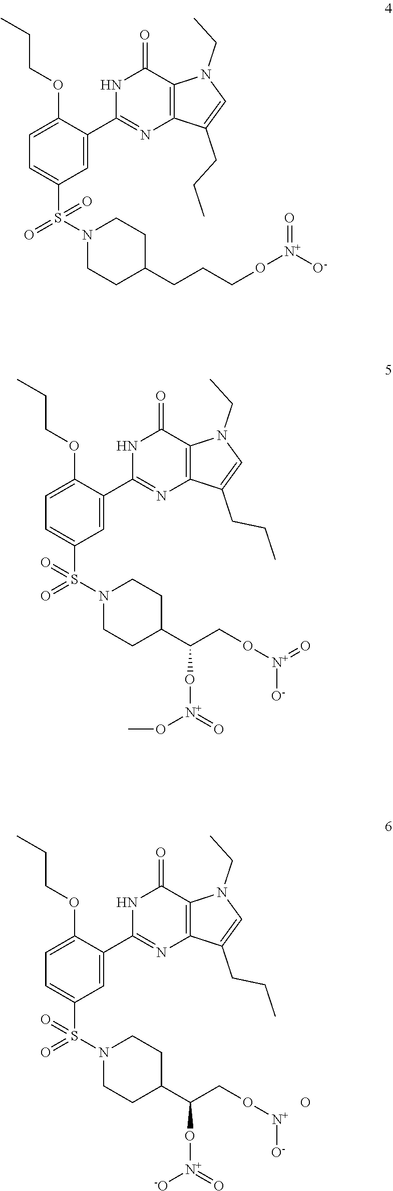

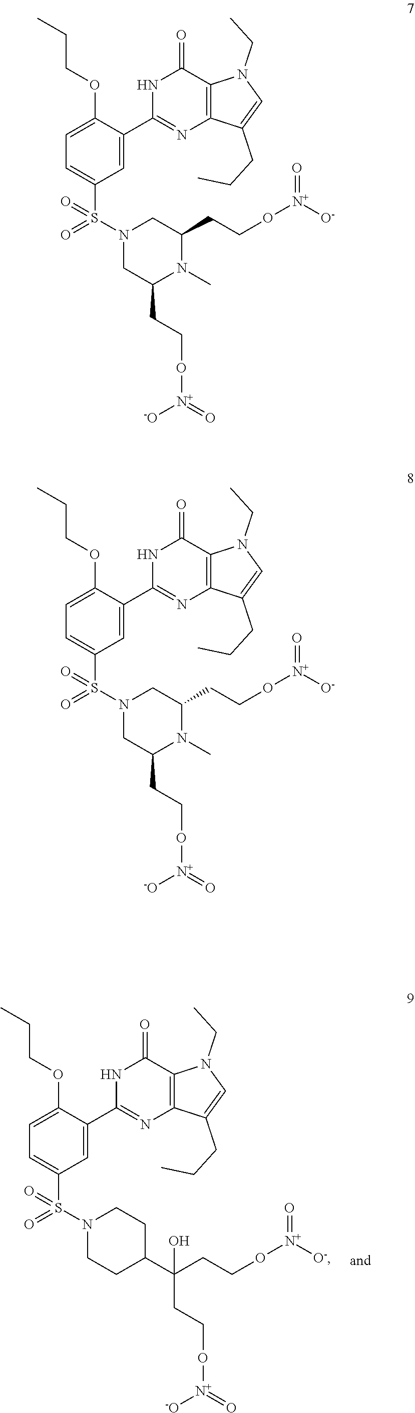

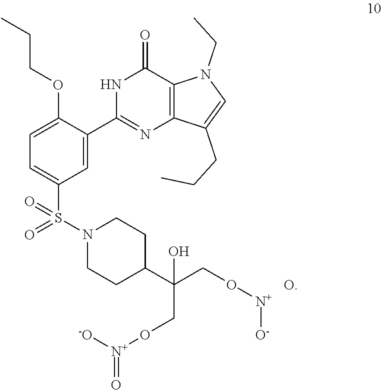

5. The topical composition of any one of the claims 1 to 3, wherein said compound of formula I is a compound selected from
(1-((3-(5-ethyl-4-oxo-7-propyl-4,5-dihydro-3H-pyrrolo[3,2-d]pyrimidin-2-yl)-4-propoxyphenyl)sulfonyl)piperidine-4,4-diyl)bis(ethane-2,1-diyl) dinitrate (1);
2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl nitrate (2);
2-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethyl nitrate (3);
3-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)propyl nitrate (4);
(R)-1-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethane-1,2-diyl dinitrate (5);
(S)-1-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)ethane-1,2-diyl dinitrate (6);
((2R,6S)-4-((3-(5-ethyl-4-oxo-7-propyl-4,5-dihydro-3H-pyrrolo[3,2-d]pyrimidin-2-yl)-4-propoxyphenyl)sulfonyl)-1-methylpiperazine-2,6-diyl)bis(ethane-2,1-diyl) dinitrate (7);
((2S,6S)-4-((3-(5-ethyl-4-oxo-7-propyl-4,5-dihydro-3H-pyrrolo[3,2-d]pyrimidin-2-yl)-4-propoxyphenyl)sulfonyl)-1-methylpiperazine-2,6-diyl)bis(ethane-2,1-diyl) dinitrate (8);
3-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)-3-hydroxypentane-1,5-diyl dinitrate (9); and
2-(1-((3-(5-ethyl-4-oxo-7-propyl-4,5-dihydro-3H-pyrrolo[3,2-d]pyrimidin-2-yl)-4-propoxyphenyl)sulfonyl)piperidin-4-yl)-2-hydroxypropane-1,3-diyl dinitrate (10).

6. The topical composition of claim of any one of the claims 1 to 5, wherein said compound of formula I is Compound 1 or Compound 2, and wherein preferably said compound of formula I is Compound 1

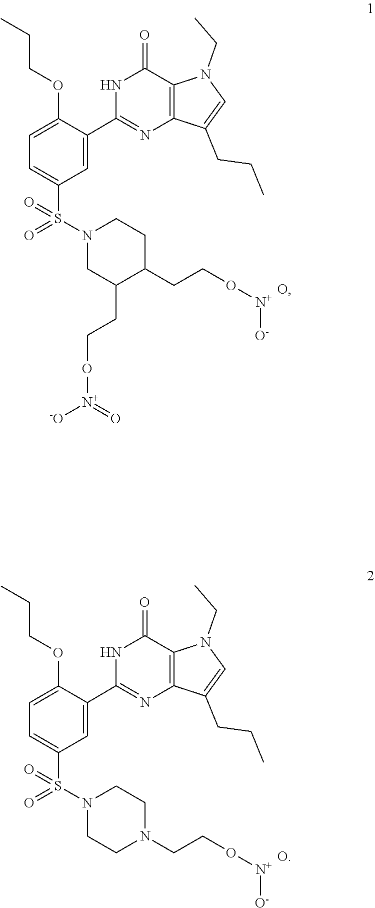

7. The composition of any one of the claims 1 to 6, wherein said compound of formula I, preferably said Compound 1, is present in said topical composition from about 0.0001% (w/v) to about 0.02% (w/v), preferably from about 0.0002% (w/v) to about 0.02% (w/v), and further preferably from about 0.0002% (w/v) to about 0.01% (w/v).

8. The composition of any one of the claims 1 to 7, wherein said compound of formula I, preferably said Compound 1, is present in said topical composition from about 0.0002% (w/v) to about 0.008% (w/v), preferably from about 0.0002% (w/v) to about 0.006% (w/v), and again further preferably from about 0.0002% (w/v) to about 0.005% (w/v), and again further preferably from about 0.0002% (w/v) to about 0.004% (w/v).

9. The topical composition of any one of the preceding claims, wherein said topical composition comprises at least one solvent, wherein said solvent is selected from the group consisting of PEG 400, PEG 350, PEG 240, ethanol, 2-(2-ethoxyethoxy)ethanol, glycerin, propylene glycol, polypropylene glycol, poloxamer 407, poloxamer 188, water, and mixtures thereof.

10. The topical composition of any one of claims 1 to 8, wherein said topical composition comprises a combination of solvents, wherein said combination of solvents comprises PEG 400 and ethanol.

11. The topical composition of any one of the preceding claims, wherein said compound of formula I is Compound 1, 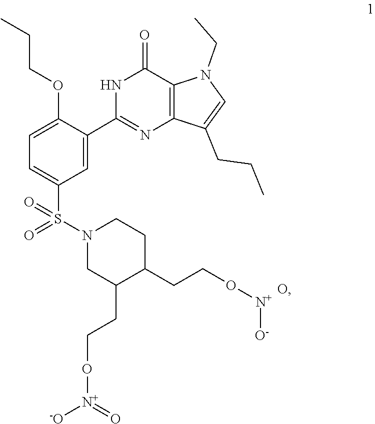
and
wherein said topical composition comprises said Compound 1 in a concentration between about 1.5 μM and about 300 μM, preferably between about 3 μM and about 200 μM, further preferably between about 5 μM and about 100 μM; and
wherein said topical composition comprises a solvent; and wherein said solvent is a combination of PEG 400 and ethanol, and wherein the ratio (vol/vol) of said PEG 400 and ethanol is from about 60:40 to 75:25, preferably from about 65:35 to 75:25, further preferably from about 68:32 to 72:28, and again further preferably about 70:30; or wherein said solvent is a combination of PEG 400, ethanol, 2-(2-ethoxyethoxy)ethanol and water, wherein the ratio (vol/vol) of said PEG400:ethanol:2-(2-ethoxyethoxy)ethanol:water is from about 5:20:5:30 to 15:40:15:70, preferably from about 8:25:8:40 to about 12:35:12:60, and further preferably about 10:30:10:50.

12. The topical composition of any one of the claims 1 to 11 for use in a method of topically treating a disease or disorder mediated by PDE5 activity and/or NO related endothelial dysfunction, preferably of a disease or disorder of the skin mediated by PDE5 activity and/or NO related endothelial dysfunction, in a subject, preferably in a human.

13. The topical composition of any one of the claims 1 to 11 for use in a method of topically treating a disease or disorder in a subject, preferably in a human, wherein said topically treating of said disease or disorder is selected from
(a) treating a skin disease, preferably skin aging or steroid-induced skin atrophy;
(b) treating erectile dysfunction;
(c) preventing or treating hair loss, or
(d) promoting hair growth via
(i) inducing anagen of hair follicles;
(ii) promoting melanogenesis;
(iii) increasing the number of hair follicles;
(iv) increasing skin thickness;
(v) promoting angiogenesis;
(vi) proliferating cells in outer root sheath; and/or
(vii) enhancing blood flow in the hair follicle.

14. The topical composition for use according to claim 12 or claim 13, wherein said topically treating of said disease or disorder is preventing or treating hair loss, wherein said hair loss is alopecia, and wherein preferably said alopecia is androgenetic alopecia (AGA) or chemotherapy-induced alopecia (CIA) or alopecia areata.

15. The topical composition for use according to claim 12 or claim 13, wherein said topically treating of said disease or disorder is promoting hair growth via
(i) inducing anagen of hair follicles;
(ii) promoting melanogenesis;
(iii) increasing the number of hair follicles;
(iv) increasing skin thickness;
(v) promoting angiogenesis;
(vi) proliferating cells in outer root sheath; and/or
(vii) enhancing blood flow in the hair follicle.
